(19) United States
(12) Patent Application Publication
(10) Pub. No.: US 2026/0060640 A1
(43) Pub. Date: Mar. 5, 2026

(54) MAMMOGRAPHY IMAGE PROCESSING APPARATUS AND ASSOCIATED METHODS

(71) Applicant: GE Precision Healthcare LLC, Waukesha, WI (US)

(72) Inventors: Laurence Vancamberg, Poissy (FR); Razvan Iordache, Clamart (FR); Pablo Milioni de Carvalho, Chaville (FR); Tiphaine Lecomte, Buc (FR); Richard Bertranine, Malakoff (FR)

(21) Appl. No.: 18/821,551

(22) Filed: Aug. 30, 2024

(51) Int. Cl. A61B 8/08; A61B 8/00; G16H 30/40; G16H 40/67

(52) U.S. Cl. CPC A61B 8/0825; A61B 8/406; A61B 8/461; A61B 8/481; G16H 30/40; G16H 40/67

(57) ABSTRACT

Systems, apparatus, articles of manufacture, and methods are disclosed that to configure a first medical imaging device, capture a first mammographic image of a patient using the first medical imaging device, analyze the first medical image, determine, in response to an analysis of the first medical image, to capture a second medical image, configure at least one of the first medical imaging device or a second medical imaging device for the second medical image different from the first medical image, capture the second medical image, and transmit the first medical image and the second medical image to an external device for processing and generating a next action with respect to the patient.

### FIELD OF THE DISCLOSURE

This disclosure relates generally to medical device operation and, more particularly, to performing breast image processing and associated methods.

### BACKGROUND

In recent years, technologists have used medical imaging devices to take breast images of patients in hospital settings. Such breast images are interpreted by a radiologist to determine if there is a likelihood that the patient has cancer, is cancer free, or if more breast images, from mammography or other imaging modalities are required to increase an accuracy of predicting if a patient does or does not have cancer.

### BRIEF DESCRIPTION OF THE DRAWINGS

FIG. 1A illustrates an example medical imaging device that is shown in a rotated position.

FIG. 1B illustrates a first example medical imaging device and a second example medical imaging device that are operating in a first hospital.

FIG. 1C illustrates an example networked infrastructure including a plurality of medical imaging devices located in different hospitals.

FIG. 1D illustrates an example networked infrastructure including a plurality of medical imaging devices in communication with breast imaging protocol circuitry and breast imaging analysis circuitry.

FIG. 2 is a block diagram of an example implementation of medical device circuitry of the medical imaging device of FIG. 1A, where the medical device circuitry is shown in communication with breast imaging processing circuitry.

FIG. 3 is a block diagram of an example implementation of image generator circuitry of the medical device circuitry of FIG. 2.

FIG. 4 is a block diagram of an example implementation of image processor circuitry of the medical device circuitry of FIG. 2.

FIG. 5 is a flowchart representative of example machine readable instructions and/or example operations that may be executed, instantiated, and/or performed by example programmable circuitry to implement the medical device circuitry of FIG. 2.

FIG. 6A is a first portion of a flowchart representative of example machine readable instructions and/or example operations that may be executed, instantiated, and/or performed by example programmable circuitry to implement the medical device circuitry of FIG. 2.

FIG. 6B is a second portion of a flowchart representative of example machine readable instructions and/or example operations that may be executed, instantiated, and/or performed by example programmable circuitry to implement the medical device circuitry of FIG. 2.

FIG. 7 is a flowchart representative of example machine readable instructions and/or example operations that may be executed, instantiated, and/or performed by example programmable circuitry to implement the medical device circuitry of FIG. 2.

FIG. 8 is a flowchart representative of example machine readable instructions and/or example operations that may be executed, instantiated, and/or performed by example programmable circuitry to implement the breast imaging processing circuitry of FIG. 2.

FIG. 9 is a flowchart representative of example machine readable instructions and/or example operations that may be executed, instantiated, and/or performed by example programmable circuitry to implement the medical device circuitry 250 of FIG. 2.

FIG. 10 is a block diagram of an example processing platform including programmable circuitry structured to execute, instantiate, and/or perform the example machine readable instructions and/or perform the example operations of FIGS. 5-9 to implement the medical device circuitry 250 of FIG. 2 and/or the breast imaging processing circuitry of FIG. 2.

FIG. 11 is a block diagram of an example implementation of the programmable circuitry of FIG. 10.

FIG. 12 is a block diagram of another example implementation of the programmable circuitry of FIG. 10.

FIG. 13 is a block diagram of an example software/firmware/instructions distribution platform (e.g., one or more servers) to distribute software, instructions, and/or firmware (e.g., corresponding to the example machine readable instructions of FIGS. 5-9) to client devices associated with end users and/or consumers (e.g., for license, sale, and/or use), retailers (e.g., for sale, re-sale, license, and/or sub-license), and/or original equipment manufacturers (OEMs) (e.g., for inclusion in products to be distributed to, for example, retailers and/or to other end users such as direct buy customers).

In general, the same reference numbers will be used throughout the drawing(s) and accompanying written description to refer to the same or like parts. The figures are not necessarily to scale.

### DETAILED DESCRIPTION

In breast imaging, a team that includes at least a technologist and a radiologist works together to diagnose and treat patients that may have cancer. The technologist is to operate medical imaging devices to generate medical images that include patient breasts. The radiologist interprets the medical images to determine if there is a likelihood that the patient has cancer, is cancer free, or if more medical images are required to increase an accuracy of predicting if a patient does or does not have cancer.

In current techniques, a technologist operates the medical imaging device to generate a medical image before transmitting the medical image to the radiologist. In this current technique, the radiologist then decides that more images are necessary, and transmits an instruction back to the technologist. While the technologist and the radiologist are communicating the medical images, which use processor cycles, the patient is waiting. The techniques described herein improve an efficiency of the process by allowing the medical imaging device to determine when to capture additional images and which medical images types to capture (e.g., mammographic X-ray, magnetic resonance imaging (MRI), ultrasound, positron emission tomography (PET), single photon emission tomography (SPECT), etc.). The medical imaging device is to follow an image capture protocol that can include different triggers (e.g., decisions) to automatically determine to generate subsequent breast images.

Generation of the correct medical images is important to ensure accuracy and timeliness, particularly while a patient and technologist are at the imaging device in an appointment. The examples disclosed herein relate to breast imaging processing circuitry to obtain and process medical images and additional images and/or patient data to drive actions in patient diagnosis and treatment. For example, the breast imaging processing circuitry can process an initial medical image to determine whether one or more additional images is to be obtained for further analysis with respect to a diagnosis or treatment of the patient.

Techniques disclosed herein relate to enabling the medical imaging device to increase interactions between devices (e.g., imaging systems, processors, etc.) and reduce interaction with a radiologist and other personnel. By reducing interactions, a technologist that operates the medical imaging device works independently from input from the radiologist for longer periods of time and much processing, configuration, and analysis is done by processing circuitry, rather than humans.

In some examples, there is a shortage of radiologists and technologists, which increases a need for medical imaging devices (e.g., automated medical imaging devices) that do not use significant amounts of input from the radiologists and technologists. Additionally, certain examples provide improved medical imaging devices, which use an image capture protocol to automatically determine whether sufficient images have been obtained to diagnose a patient. If more image(s) are warranted, the medical imaging devices then communicate and configure to obtain more images for diagnosing the patient in accordance with the image capture protocol. If sufficient image(s) have been obtained, then the devices package and transmit the sufficient images for display, processing and analysis to drive a next action in care of the patient (e.g., treatment, scheduling a follow-up visit, other care plan, etc.).

One type of mammographic image is a contrast-enhanced mammography (CEM) image. A CEM image can be generated at various times during examination of a patient. For example, in contrast-enhanced mammography, a contrast agent (e.g., iodine) is injected into a patient's arm. After injection into the arm of the patient, the fibroglandular tissue of the breast absorbs the contrast agent, which takes an amount of time (e.g., two minutes after injection). A first CEM image or a first set of CEM images may be acquired after a first time period (e.g., the time for the fibroglandular tissue absorbs the contrast agent such as two minutes, etc.). The first set of CEM images may include, for either one or multiple breasts, a cranio-caudal (CC) view and a medio lateral oblique (MLO) view. In some examples, the first set of CEM image or first set of CEM images is captured with a dual energy technique.

In some examples, there is a second CEM image or a second set of CEM images. In such examples, the second CEM image is referred to as a late CEM image or a subsequent mammographic image. In some examples, the second set of CEM images (e.g., referred to as late CEM images) includes, for either one or multiple breasts, at least one of a second cranio-caudal (CC) view, a second medio lateral oblique (MLO) view, or a first medio lateral (ML) view. The second set of CEM images is captured after a second time period (e.g., five minutes after the first CEM image set is acquired).

As such, certain examples determine whether an additional image, such as a late CEM image, etc., should be acquired in an exam and/or whether another exam (e.g., including additional images of a same modality, additional images of a different modality, etc.) should be performed. In such examples, a recommendation, an order, further instructions, etc., can be generated to acquire an additional image inside the current examination, trigger a new examination, etc.

Examples disclosed herein include methods and apparatus to perform medical examinations with a plurality of imaging modalities, and, within the medical examinations, process and enhance generated medical images with one or more additional images, such as a late contrast-enhanced mammography (CEM) image, etc. Examples disclosed herein can be implemented on one or more medical imaging devices at a single location and/or can be networked across multiple locations.

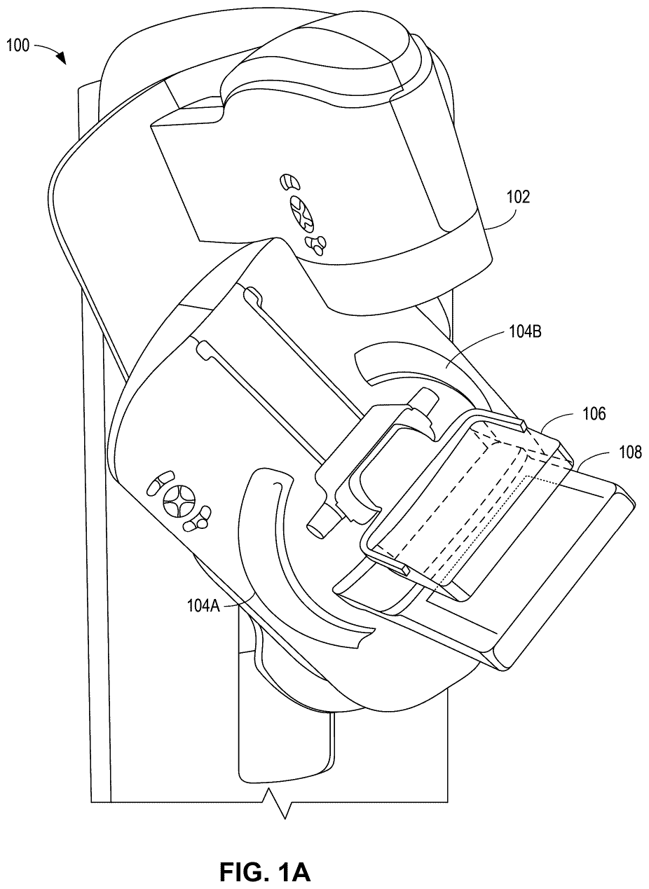

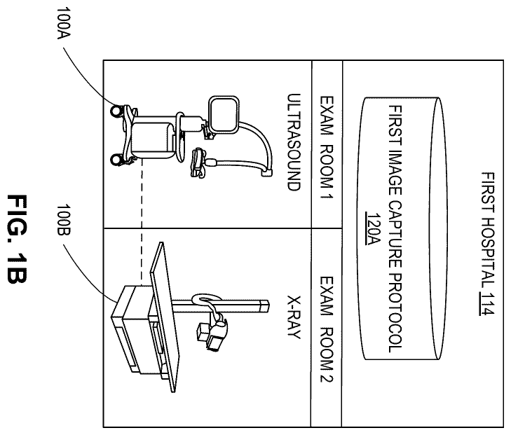

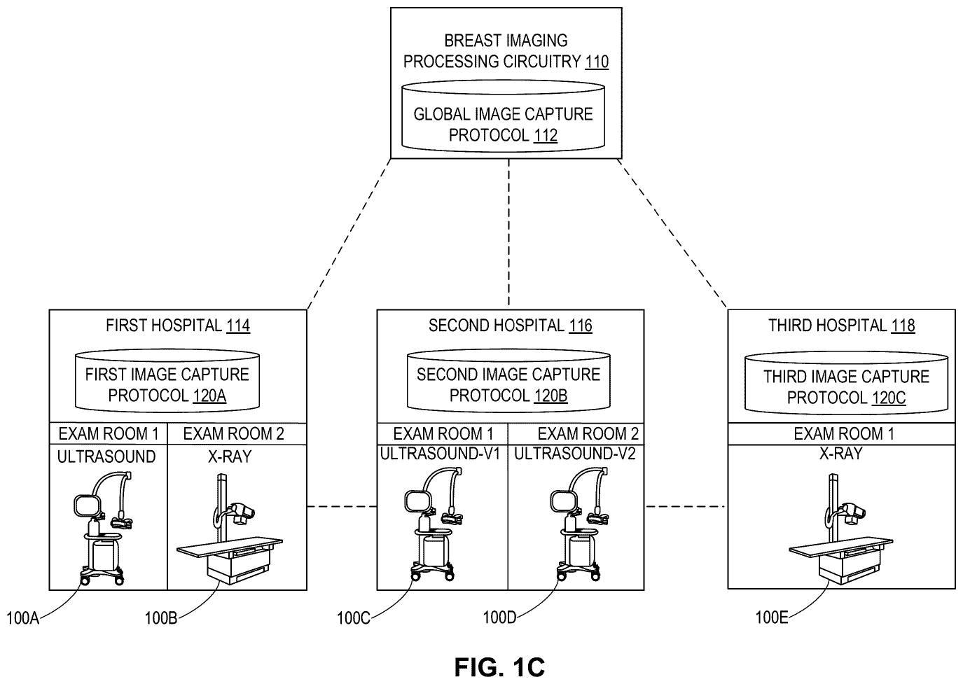

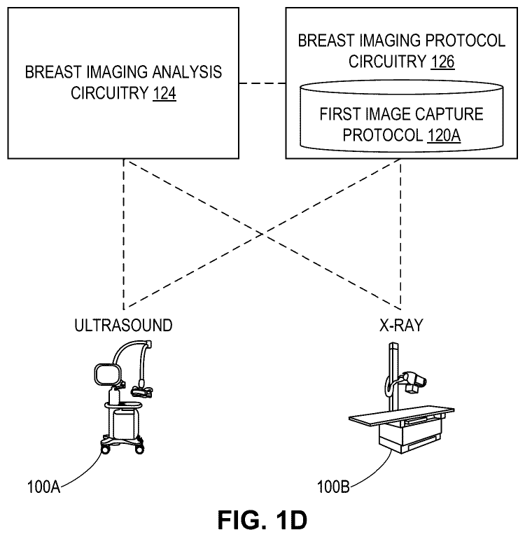

Turning to the figures, FIG. 1A shows an example medical imaging device 100 that is shown in a rotated position. The example medical imaging device 100 includes an example source 102, an example left arm bar 104A, an example right arm bar 104B, an example compression tray 106 (e.g., a compression paddle), and an example detector 108. In breast imaging, the patient places a left hand on the curved surface of the left arm bar 104A, a right hand on the curved surface of the right arm bar 104B, and breasts are compressed by the compression tray 106 and the detector 108 (e.g., which also serves as a breast support). While this example is described in the context of mammography, the machine and associated processes described herein can be applied to radiographic imaging of other objects. The source 102 (e.g., beam emitter) includes an aperture that emits a beam (X-ray, gamma ray, ultrasound, etc.) which interacts with the detector 108 (e.g., detector plate, detection surface, etc.).

In the example of FIG. 1, the detector 108 is a flat-panel detector that is located behind the object to be imaged (e.g., the breasts of the patient) which is located underneath the source 102. The beams from the source 102 interact with the inner structures of the object to be imaged, and the inner structures are represented by the relative intensity of the signals captured. Different tissues of the human body have different features which may involve different radiation dosage, resulting in varying levels of signal strength and noise in the received image data. The example medical imaging device 100 of FIG. 1A determines, in an automated manner, which types of images are to be taken for diagnosing a patient along with whether additional mammographic images are to be taken based on an image capture protocol. The example medical imaging device 100 of FIG. 1A, based on the image capture protocol, determines which modality is used for capturing the images. Note that while a mammography example is used to describe certain examples, mammography is used for purposes of illustration, and the medical imaging device 100 can be another modality such as magnetic resonance imaging (MRI), ultrasound, positron emission tomography (PET), single photon emission tomography (SPECT), etc.

FIG. 1B illustrates an example first medical imaging device 100A and an example second medical imaging device 100B that are operating in an example first hospital 114. In the example of FIG. 1B, the first hospital includes an example first image capture protocol 120A. The first image capture protocol 120A is loaded on the first medical imaging device 100A and the second medical imaging device 100B. In the example of FIG. 1B, the first medical imaging device 100A is an ultrasound machine and the second medical imaging device 100B is an X-ray machine. For example, if the first image capture protocol 120A includes instructions to take an ultrasound after an X-ray, the second medical imaging device 100B, by executing the first image capture protocol 120A, transmits an instruction to the first medical imaging device 100A that a patient has recently finished an X-ray and is coming to the first examination room for an ultrasound. After the first medical imaging device 100A receives the instruction from the second medical imaging device 100B, the first medical imaging device 100A begins to prepare to take the ultrasound images. Therefore, the first medical imaging device 100A and the second medical imaging device 100B save time by warming up the first medical imaging device 100A as the patient is moving from the second examination room to the first examination room.

FIG. 1C shows an example networked infrastructure including a plurality of medical imaging devices 100A, 100B, 100C, 100D, 100E. As shown in FIG. 1C, an example first hospital 114 includes the first medical imaging device 100A (e.g., an ultrasound device) and the second medical imaging device 100B (e.g., an X-ray machine). An example second hospital 116 includes a third medical imaging device 100C (e.g., an ultrasound machine with first components) and a fourth medical imaging device 100D (e.g., an ultrasound machine with second components). For example, the fourth medical imaging device 100D has upgraded software (e.g., software version 2.0) and/or hardware compared to the third medical imaging device 100C. An example third hospital 118 only includes a fifth medical imaging device 100E (e.g., an X-ray machine).

The example plurality of medical imaging devices 100A, 100B, 100C, 100D, 100E of the three hospitals 114, 116, 118 are shown in communication with example breast imaging processing circuitry 110. In some examples, the example breast imaging processing circuitry 110 generates a global image capture protocol which is stored in the global image capture protocol data store 112 that is used by the medical imaging devices 100A, 100B, 100C, 100D, 100E. The example global image capture protocol, which is stored in the global image capture protocol data store 112, includes instructions for various hospital setups.

For example, the first image capture protocol 120A is different from the second image capture protocol 120B due to the breast imaging processing circuitry 110 determining the devices of the first hospital 114 are different than the devices of the second hospital 116. For example, the breast imaging processing circuitry 110 determines that the first hospital 114 includes an ultrasound machine and an X-ray machine and that the second hospital 116 includes two ultrasound machines and does not include an X-ray machine. Therefore, the second image capture protocol 120B does not include protocols to take ultrasounds after X-rays (e.g., the first image capture protocol includes protocols to take ultrasounds after X-rays), but rather begins the protocols at taking ultrasounds and skips the X-ray instructions.

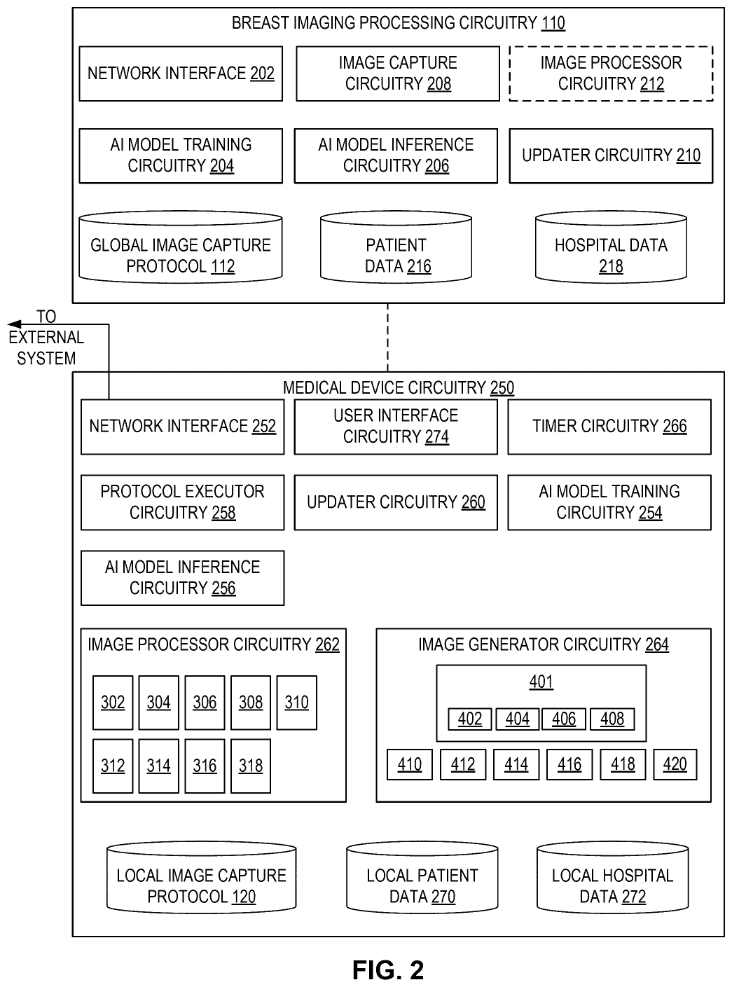

In some examples, the example breast imaging processing circuitry 110 performs image analysis at a remote location and transmits results of the image analysis back to the medical imaging devices 100A, 100B, 100C, 100D, 100E. The medical imaging devices 100A, 100B, 100C, 100D, 100E can each implement medical device circuitry 250 (FIG. 2), which is in network communication with the breast image processing circuitry 110. In some examples, the first medical imaging device 100A is in network with the other medical imaging devices 100B, 100C, 100D, 100E. In such examples, a patient is diagnosed at the third hospital 118 with the fifth medical imaging device 100E (e.g., an X-ray machine) and is instructed for a follow-up visit at the second hospital 116 which includes two ultrasound machines. The fifth medical imaging device 100E transmits the X-rays and/or radiologist notes to the breast imaging processing circuitry 110, which then transmits the X-rays and/or radiologist notes to for use by the third medical imaging device 100C and/or the fourth medical imaging device 100D. In other examples, the fifth medical imaging device 100E is to transmit the X-rays and/or radiologist notes to the third medical imaging device 100C without using the breast imaging processing circuitry 110 as an intermediary.

In some examples, the first medical imaging device 100A, after capturing a first image determines that a subsequent image (e.g., subsequent operation) is warranted. The first medical imaging device 100A then determines a type of the subsequent image. For example, the subsequent image may be of a first type that corresponds to the first image (e.g., the first image is an X-ray image and the subsequent image is also an X-ray image). Alternatively, the subsequent image may be of a second type which is different from the type of the first image (e.g., the first image is an X-ray image and the subsequent image is an ultrasound image). In this example, the first medical imaging device 100A determines the type of the subsequent image. In other examples, the first medical imaging device 100A communicates with the example breast imaging processing circuitry 110. For example, the first medical imaging device 100A transmits the first image to the breast imaging processing circuitry 110 and the breast imaging processing circuitry 110 determines that a subsequent image is warranted and transmits the instructions to capture the subsequent image to either the first medical imaging device 100A or one of the other medical imaging devices 100B, 100C, 100D, 100E.

FIG. 1D illustrates an example networked infrastructure including a plurality of medical imaging devices 100A, 100B in communication with example breast imaging analysis circuitry 124 and example breast imaging protocol circuitry 126. In the example of FIG. 1D, the functionality of the breast imaging processing circuitry 110 (FIG. 1C), is divided between the example breast imaging analysis circuitry 124 and the example breast imaging protocol circuitry 126.

The example breast imaging protocol circuitry 126 operates at an examination level (e.g., determines which exam or imaging modality to use). For example, the breast imaging protocol circuitry 126 (e.g., primary circuitry, pilot circuitry, etc.) instructs (e.g., commands, controls, operates, etc.) the plurality of medical imaging devices 100A, 100B (e.g., secondary circuitry, worker circuitry, etc.) to capture one or more medical images. In some examples, the plurality of medical imaging devices 100A, 100B (e.g., the ultrasound machine, the X-ray machine, etc.) transmit captured images for analysis to the example breast imaging analysis circuitry 124 and await further instructions from the breast imaging protocol circuitry 126. In other examples, the plurality of medical imaging devices 100A, 100B are not in communication with the example breast imaging analysis circuitry 124 and therefore transmit the captured images to the example breast imaging protocol circuitry 126, which subsequently transmits the captured images to the example breast imaging analysis circuitry 124. In some examples, the breast imaging analysis circuitry 124 operates at an image level (e.g., determines if a single image is blurry, if there are findings in the image, etc.). In some examples, the breast imaging analysis circuitry 124 operates at the examination level (e.g., there are findings determined in a set of images such as a complete examination).

In some examples, the breast imaging analysis circuitry 124 and/or the breast imaging protocol circuitry 126 is located at a remote location (e.g., a server, cloud-based server, etc.) that is not on hospital grounds. In other examples, the breast imaging analysis circuitry 124 and/or the breast imaging protocol circuitry 126 is located at an external device that is operable by a technician and on hospital grounds. In yet other examples, the breast imaging analysis circuitry 124 and/or the breast imaging protocol circuitry 126 is located on the first medical imaging device 100A.

In some examples, the breast imaging protocol circuitry 126 transmits an instruction of a specific examination to perform to one of the plurality of medical imaging devices 100A, 100B (e.g., the first medical imaging device 100A). In such examples, the first medical imaging device 100A stores the instruction of the specific examination to perform. Once the first medical imaging device 100A is activated (e.g., by a technician, by a signal from the breast imaging protocol, via instruction from another device, etc.), the first medical imaging device 100A is able to begin the specific examination.

For example, once the breast imaging protocol circuitry 126 determines that a specific patient is at the first hospital 114 (FIG. 1C), the breast imaging protocol circuitry 126 accesses (e.g., retrieves, reads, etc.) patient data from the first image capture protocol 120A to determine which examination is to be performed by one or more of the plurality of medical imaging devices 100A, 100B. In some examples, a technician opens a patient list on an external device (e.g., the first medical imaging device 100A, a workstation, a compute device, etc.) and identifies a patient, and the external device queries the breast imaging protocol circuitry 126. In such examples, the breast imaging protocol circuitry 126 determines which examination to perform for the identified patient on the patient list.

FIG. 2 is a block diagram of an example implementation of the medical device circuitry 250 of the medical imaging device 100 of FIG. 1A, where the medical device circuitry 250 is in communication with breast imaging processing circuitry 110. The example implementation of the medical device circuitry 250 of FIG. 2 captures, analyzes, and transmits mammographic images. The medical device circuitry 250 of FIG. 2 may be instantiated (e.g., creating an instance of, bring into being for any length of time, materialize, implement, etc.) by programmable circuitry such as a Central Processor Unit (CPU) executing first instructions. Additionally or alternatively, the medical device circuitry 250 of FIG. 2 may be instantiated (e.g., creating an instance of, bring into being for any length of time, materialize, implement, etc.) by (i) an Application Specific Integrated Circuit (ASIC) and/or (ii) a Field Programmable Gate Array (FPGA) structured and/or configured in response to execution of second instructions to perform operations corresponding to the first instructions. It should be understood that some or all of the circuitry of FIG. 2 may, thus, be instantiated at the same or different times. Some or all of the circuitry of FIG. 2 may be instantiated, for example, in one or more threads executing concurrently on hardware and/or in series on hardware. Moreover, in some examples, some or all of the circuitry of FIG. 2 may be implemented by microprocessor circuitry executing instructions and/or FPGA circuitry performing operations to implement one or more virtual machines and/or containers.

The example implementation of the breast imaging processing circuitry 110 of FIG. 1C generates an image capture protocol for use by the medical device circuitry 250. In some examples, different functionalities of the breast imaging processing circuitry 110 of FIG. 1C are divided into the breast imaging analysis circuitry 124 (FIG. 1D) and the breast imaging protocol circuitry 126 (FIG. 1D). The breast imaging processing circuitry 110 of FIG. 2 may be instantiated (e.g., creating an instance of, bring into being for any length of time, materialize, implement, etc.) by programmable circuitry such as a Central Processor Unit (CPU) executing first instructions. Additionally or alternatively, the breast imaging processing circuitry 110 of FIG. 2 may be instantiated (e.g., creating an instance of, bring into being for any length of time, materialize, implement, etc.) by (i) an Application Specific Integrated Circuit (ASIC) and/or (ii) a Field Programmable Gate Array (FPGA) structured and/or configured in response to execution of second instructions to perform operations corresponding to the first instructions. It should be understood that some or all of the circuitry of FIG. 2 may, thus, be instantiated at the same or different times. Some or all of the circuitry of FIG. 2 may be instantiated, for example, in one or more threads executing concurrently on hardware and/or in series on hardware. Moreover, in some examples, some or all of the circuitry of FIG. 2 may be implemented by microprocessor circuitry executing instructions and/or FPGA circuitry performing operations to implement one or more virtual machines and/or containers.

The breast imaging processing circuitry 110 includes a network interface 202, artificial intelligence (AI) model training circuitry 204, AI model inference circuitry 206, image capture circuitry 208, updater circuitry 210, a global image capture protocol data store 112, a patient data store 216, and a hospital data store 218. In some examples, as illustrated by the dashed lines, the breast imaging processing circuitry 110 includes image processor circuitry 212. The medical device circuitry 250 includes a network interface 252, AI model training circuitry 254, AI model inference circuitry 256, protocol executor circuitry 258, updater circuitry 260, image processor circuitry 262, image generator circuitry 264, timer circuitry 266, a local image capture protocol data store 120, a local patient data store 270, a local hospital data store 272, and user interface circuitry 274.

While there are numerous components in the breast imaging processing circuitry 110 and the medical device circuitry 250, the example AI model training circuitry 204, the example AI model inference circuitry 206, the example AI model training circuitry 254, the example AI model inference circuitry 256 will be discussed together before returning to the discussion of the other components of the breast imaging processing circuitry 110 and the medical device circuitry 250.

Artificial intelligence (AI), including machine learning (ML), deep learning (DL), and/or other artificial machine-driven logic, enables machines (e.g., computers, logic circuits, etc.) to use a model to process input data to generate an output based on patterns and/or associations previously learned by the model via a training process. For instance, the model may be trained with data to recognize patterns and/or associations and follow such patterns and/or associations when processing input data such that other input(s) result in output(s) consistent with the recognized patterns and/or associations.

In general, implementing a ML/AI system involves two phases, a learning/training phase and an inference phase. In the learning/training phase, a training algorithm is used to train a model to operate in accordance with patterns and/or associations based on, for example, training data. In general, the model includes internal parameters that guide how input data is transformed into output data, such as through a series of nodes and connections within the model to transform input data into output data. Additionally, hyperparameters are used as part of the training process to control how the learning is performed (e.g., a learning rate, a number of layers to be used in the machine learning model, etc.). Hyperparameters are defined to be training parameters that are determined prior to initiating the training process.

Different types of training may be performed based on the type of ML/AI model and/or the expected output. For example, supervised training uses inputs and corresponding expected (e.g., labeled) outputs to select parameters (e.g., by iterating over combinations of select parameters) for the ML/AI model that reduce model error. As used herein, labelling refers to an expected output of the machine learning model (e.g., a classification, an expected output value, etc.) Alternatively, unsupervised training (e.g., used in deep learning, a subset of machine learning, etc.) involves inferring patterns from inputs to select parameters for the ML/AI model (e.g., without the benefit of expected (e.g., labeled) outputs).

In examples disclosed herein, ML/AI models are trained using stochastic gradient descent. However, any other training algorithm may additionally or alternatively be used. In examples disclosed herein, training is performed until an acceptable amount of error is achieved. In examples disclosed herein, training is performed at either a local medical imaging device (e.g., the first medical imaging device 100A of FIG. 1C) or remotely at a central facility (e.g., the breast imaging processing circuitry 110 of FIG. 1C). Training is performed using hyperparameters that control how the learning is performed (e.g., a learning rate, a number of layers to be used in the machine learning model, etc.).

Training is performed using training data. In examples disclosed herein, the training data originates from publicly available data, locally generated data (e.g., previous patient images). Because supervised training is used, the training data is labeled. Labeling is applied to the training data by a radiologist with knowledge of different mammographic images or a data scientist with knowledge of different outputs.

Once training is complete, the model is deployed for use as an executable construct that processes an input and provides an output based on the network of nodes and connections defined in the model. The model is stored at the AI model training circuitry 204 of the breast imaging processing circuitry 110 and/or the by the AI model training circuitry 254 of the medical device circuitry 250. The model may then be executed by the AI model inference circuitry 206 of the breast imaging processing circuitry 110 and/or by the AI model inference circuitry 256 of the medical device circuitry 250.

Once trained, the deployed model may be operated in an inference phase to process data. In the inference phase, data to be analyzed (e.g., live data) is input to the model, and the model executes to create an output. This inference phase can be thought of as the AI “thinking” to generate the output based on what it learned from the training (e.g., by executing the model to apply the learned patterns and/or associations to the live data). In some examples, input data undergoes pre-processing before being used as an input to the machine learning model. Moreover, in some examples, the output data may undergo post-processing after it is generated by the AI model to transform the output into a useful result (e.g., a display of data, an instruction to be executed by a machine, etc.).

In some examples, output of the deployed model may be captured and provided as feedback. By analyzing the feedback, an accuracy of the deployed model can be determined. If the feedback indicates that the accuracy of the deployed model is less than a threshold or other criterion, training of an updated model can be triggered using the feedback and an updated training data set, hyperparameters, etc., to generate an updated, deployed model.

The example AI model training circuitry 204 performs training of an artificial intelligence model that is used to at least one of generate an image capture protocol and/or perform analysis of the images generated during execution of the image capture protocol. An example AI model can be trained on patient data stored in the patient data store 216, trained on hospital data (e.g., the number and availability of machines stored in the hospital) in the hospital data store 218, and trained on various iterations of the image capture protocol that are stored in the global image capture protocol data store 112. After the AI model training circuitry 204 trains the AI model, the AI model is referred to as a trained AI model.

The example AI model inference circuitry 206 executes the trained AI model. By executing the trained AI model, the AI model inference circuitry 206 generates predictions and recommendations of different mammographic images that are to be captured by the medical imaging devices 100A, 100B, 100C, 100D, 100E that are situated in the various hospitals 114, 116, 118.

The example AI model training circuitry 204 and the example AI model inference circuitry 206 train, deploy, and inference on at least two models: a pertinence score model and a medical image model. The pertinence score model receives, as an input, the medical examinations already performed. Based on the examinations already performed, the pertinence score model queries pre-determined protocol preferences of the hospital. Based on the query of the pre-determined protocol preferences of the hospital (e.g., the medical devices available, etc.), the pertinence score model determines a list of new medical examinations to be performed which is sorted by a pertinence score (e.g., relevancy score, suitability score, etc.). As used herein, a pertinence score defines a usefulness of a medical examination for a specific patient. The pertinence score model is trained to provide pertinence scores based on training data and relationships including survival rates, examination accuracy, radiology society guidelines, etc., for different possible examination pathways to diagnose a disease and/or other patient condition. The pertinence score model, once executed by the AI model inference circuitry 206, chooses the medical examination with a highest pertinence score and availability in the current hospital or other healthcare environment to suggest the next examination/action to perform.

The medical image model operates in conjunction with the pertinence score model and analyzes images of the current examination. The medical image model analyzes images of the current examination and modifies the pertinence score of the listed exams to suggest (e.g., select, choose, indicate, etc.) the medical examination with a highest pertinence score and availability in the current hospital/healthcare environment as the next examination to perform. The AI model training circuitry 204 trains the medical image model on medical images. The medical image model detects image characteristics and then correlates the image characteristics with known survival rates, examination accuracy, radiology society guidelines, etc., as well as a list of the examinations that have already been performed. The medical image model, once executed by the AI model inference circuitry 206, provides new pertinence scores to update the previous list of pertinence scores. The updated pertinence scores are used to improve (e.g., optimize) the examination pathway used to diagnose the disease and/or other condition, for example.

The example AI model training circuitry 254 is substantially similar to the AI model training circuitry 204 of the breast imaging processing circuitry 110. The AI model training circuitry 254 trains AI models that are used in computer aided detection and image processing. The AI model training circuitry 254 trains AI models with previous results from inferences performed by the AI model inference circuitry 256.

The AI model inference circuitry 256 is substantially similar to the AI model inference circuitry 206 of them of the breast imaging processing circuitry 110. In some examples, the AI model inference circuitry 256 is used as a substitute for computer-aided detection (CAD). In such examples, the AI model inference circuitry 256 implements an artificial intelligence model to determine if an image is suspicious. The results in the feedback from the inference, if reviewed by a radiologist or analyzed by an external system, may be used by the AI model training circuitry 254 to perform tuning and/or training of the AI model.

Returning to the discussion of the breast imaging processing circuitry 110, the example network interface 202 transmits the image capture protocol to the medical device circuitry 250 which can be implemented on the medical imaging device 100 of FIG. 1A (e.g., implemented on any of the medical imaging devices 100A, 100B, 100C, 100D, 100E of FIG. 1C). The network interface 202 of the breast imaging processing circuitry 110 accesses individual hospitals such as the first hospital 114 in a plurality of hospitals 114, 116, 118. In the example of FIG. 1, the hospitals 114, 116, 118 have different medical imaging devices 100A, 100B, 100C, 100D, 100E available for capturing medical images. The hospitals 114, 116, 118 transmit (e.g., report, send, transfer, etc.) information regarding the hospitals to the network interface 202 of the breast imaging processing circuitry 110. The example network interface 202 then stores the information in the hospital data store 218. In some examples, the network interface 202 receives patient data and/or patient images. After receiving patient data and/or images, the network interface 202 stores the patient data (e.g., demographic patient data, medical history data, etc.) and/or the images in the patient data store 216. The example breast imaging processing circuitry 110 uses the network interface 202 to transmit a global image capture protocol from the global image capture protocol data store 112.

The example image capture circuitry 208 generates the image capture protocol. The image capture circuitry 208 generates the image capture protocol based on hospital data that is stored in the hospital data store 218. In some examples, the image capture circuitry 208 generates a global image capture protocol that can be downloaded by any of the medical imaging devices 100A, 100B, 100C, 100D, 100E that are operating in the hospitals 114, 116, 118. In some examples, the image capture circuitry 208 selects an image capture protocol (e.g., the first image capture protocol 120A of FIG. 1C) from a plurality of image capture protocols and transmits the selected image capture protocol to a selected hospital (e.g., the first hospital 114 of FIG. 1C). In some examples, the example image capture circuitry 208 generates the image capture protocol by determining which medical imaging devices 100A, 100B, 100C, 100D, 100E are available for use in the hospitals 114, 116, 118 (e.g., the first hospital 114 is using the first medical imaging device 100A and the second medical imaging device 100B). In some examples, the breast imaging protocol circuitry 126 (FIG. 1D) generates and transmits the image capture protocol, which is used to operate the medical imaging devices 100A, 100B, 100C, 100D, 100E from a remote location.

The example updater circuitry 210 updates the image capture protocol based on data sourced at a specific hospital. For example, a second hospital 116 does not include an X-ray machine. In such examples, the updater circuitry 210 does not execute a portion of the image capture protocol that corresponds to taking mammographic images with an X-ray in the image capture protocol. In some examples, the image capture circuitry 208 is to instruct the medical device circuitry 250 to generate mammographic images. In some examples, the updater circuitry 210 dynamically evaluates a configuration of medical imaging devices 100A, 100B, 100C, 100D, 100E at the hospitals 114, 116, 118 on an ongoing basis (e.g., once a day, once a month, once a year, etc.). In other examples, the updater circuitry 210 is configured initially and updates the image capture protocol based on information that is received (e.g., new software capability, new model, corrected model, bug fix, etc.). In some examples, the breast imaging protocol circuitry 126 (FIG. 1D) updates the image capture protocols which are stored in the first image capture protocol data store 120A (FIG. 1D).

In some examples, the breast imaging processing circuitry 110 includes image processor circuitry 212. The image processor circuitry 212 is substantially similar to the image processor circuitry 262 of the medical device circuitry 250. In some examples, where the breast imaging processing circuitry 110 includes image processor circuitry 212, the medical imaging devices 100A, 100B, 100C, 100D, 100E generate medical images, transmit the medical images to the breast imaging processing circuitry 110, which then analyzes the images before making a recommendation for subsequent images to be captured by the medical imaging devices 100A, 100B, 100C, 100D, 100E. In some examples, the breast imaging analysis circuitry 124 analyzes the images and reports the results of the analysis to the breast imaging protocol circuitry 126. In such examples, the breast imaging protocol circuitry 126, after receiving the results from the breast imaging analysis circuitry 124, determines a recommendation for subsequent images to be captured.

Medical device circuitry 250 is circuitry that is used to operate any of the medical imaging devices 100A, 100B, 100C, 100D, 100E. In the example of FIG. 1, the medical imaging devices 100A, 100B, 100C, 100D, 100E include ultrasound machines (e.g., the first medical imaging device 100A, the third medical imaging device 100C, and the fourth medical imaging device 100D) and X-ray machines (e.g., the second medical imaging device 100B, the fifth medical imaging device 100E). However, in other examples, other medical imaging devices can implement the medical device circuitry 250. For ease of description, the medical device circuitry 250 refers to a medical device that includes functionality of an ultrasound machine and an X-ray machine. However, in some examples, if the medical device circuitry 250 is implemented on an ultrasound machine, the functionality of the X-ray machine is not present. Alternatively, in other examples, if the medical device circuitry 250 is implemented on an X-ray machine, then the functionality of the ultrasound machine is not present.

The example network interface 252 is substantially similar to the network interface 202 of the breast imaging processing circuitry 110. By generating multiple mammographic images without sending a transmission to a radiologist or an external radiology system requesting further images, the medical device circuitry 250 saves processor cycles by transmitting at least two mammographic images at one time. By using the image capture protocol, the medical device circuitry 250 determines which mammographic images are to be captured based on analysis of previously captured images. As used herein, an external radiology system is a compute device that is able to display images to a user. The external radiology system includes image processing capabilities and may transmit instructions without input from the radiologist.

For example, if a first X-ray is taken of the breasts of the patient, and an example image capture protocol includes an operation to evaluate the blurriness (e.g., sharpness, clarity, etc.) of the mammographic image, then the medical device circuitry 250 determines if the first X-ray is clear. In such examples, if the medical device circuitry 250 determines that the first X-ray is not clear, the example medical device circuitry 250 configures the medical imaging device 100 to take a second X-ray (e.g., subsequent mammographic image) which has an increased clarity compared to the first X-ray and indicates to a technologist to take the second X-ray (e.g., retake the first X-ray image). The medical device circuitry 250 then transmits the clearer, second X-ray to the radiologist or the external radiology system. By transmitting the second X-ray to the radiologist or the external radiology system, the medical device circuitry 250 efficiently uses processor resources by not using processor resources to send an inferior first image to the external radiology system, then receive an instruction from the external radiology system to take a second image, and then send the second image to the external radiology system.

The protocol executor circuitry 258 directs the image generator circuitry 264 to generate various mammographic images and to direct the image processor circuitry 262 to perform analysis on the generated images. By directing the image generator circuitry 264 and the image processor circuitry 262, the protocol executor circuitry 258 follows the guidelines (e.g., decision pathways, protocols, instructions, etc.) of the image capture protocol that is stored in the local image capture protocol data store 120.

The updater circuitry 260 adapts the image capture protocol based on hospital information (e.g., medical device availability, etc.). For example, if a first instance of the medical device circuitry 250 is implemented on the third medical imaging device 100C of FIG. 1C (e.g., the ultrasound machine) and a second instance of the medical device circuitry 250 is implemented on the fourth medical imaging device 100D of FIG. 1C (e.g., the ultrasound machine), the updater circuitry 260 then removes references to medical imaging devices that are not available in the second hospital 116 (FIG. 1) in the image capture protocol. In this example, the updater circuitry 260 removes references to X-ray machines in the image capture protocol.

In some examples, the updater circuitry 260 adds different operations if the local image capture protocol initially does not refer to medical imaging devices that are available. For example, in the first hospital 114 of FIG. 1, there is a first medical imaging device 100A (e.g., an ultrasound machine) and a second medical imaging device 100B (e.g., an X-ray machine). If a third instance of the medical device circuitry 250 is implemented on the first medical imaging device 100A of a first type (e.g., ultrasound machine), and the local image capture protocol stored (e.g., the first image capture protocol 120A) in the local image capture protocol data store 120 does not include operations of a second medical imaging device 100B of a second type (e.g., X-ray machine), the updater circuitry 260, after verification from the network interface 252, adapts the local image capture protocol to include operations that allow for usage of the second medical imaging device 100B (e.g., the X-ray machine). For example, the updater circuitry 260 reads the first image capture protocol 120A and determines that the instructions regarding using a second medical imaging device 100B are to be included. The updater circuitry 260 modifies the current first image capture protocol 120A to include the instructions regarding using a second medical imaging device 100B. The updater circuitry 260 receives the instructions regarding a second medical imaging device 100B from the global image capture protocol data store 112 of the breast imaging processing circuitry 110, for example.

The local image capture protocol data store 120 is implemented by any memory, storage device and/or storage disc for storing data such as, for example, flash memory, magnetic media, optical media, solid state memory, hard drive(s), thumb drive(s), etc. Furthermore, the data stored in the local image capture protocol data store 120 may be in any data format such as, for example, binary data, comma delimited data, tab delimited data, structured query language (SQL) structures, etc. While, in the illustrated example, the local image capture protocol data store 120 is illustrated as a single device, the local image capture protocol data store 120 and/or any other data storage devices described herein may be implemented by any number and/or type(s) of memories.

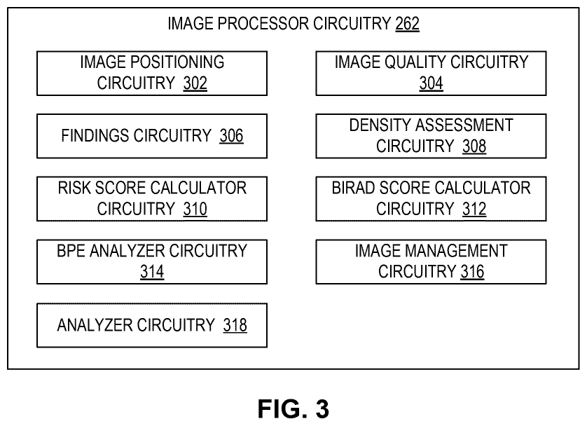

The image processor circuitry 262 performs analysis of images captured by the medical device circuitry 250. The image processor circuitry 262 includes various subcomponents (e.g., image positioning circuitry 302, image quality circuitry 304, findings circuitry 306, density assessment circuitry 308, risk score calculator circuitry 310, Breast-Imaging Reporting and Data System (BIRADS) score calculator circuitry 312, Background Parenchymal Enhancement (BPE) analyzer circuitry 314, image management circuitry 316, and analyzer circuitry 318, etc.) that are further discussed in connection with FIG. 3. The example image processor circuitry 262 uses the various sub-components in performing such analysis. By performing analysis of breast images, the image processor circuitry 262 is to output a recommendation if further breast images are to be generated (e.g., taken, capture, etc.) by the image generator circuitry 264. In some examples, results from a first imaging modality may be combined with results from a second imaging modality. For example, a BPE level, as analyzed by the BPE analyzer circuitry 314, is used for a contrast enhanced magnetic resonance imaging (MRI) image. For example, findings from the findings circuitry 306 and scores from the BIRADS score calculator circuitry 312 are used for all the types of imaging modalities. For example, an MRI specific analysis is related to contrast agent uptake kinetics analysis.

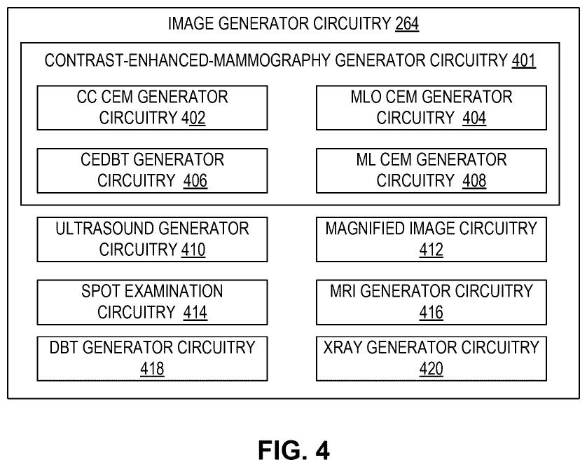

The image generator circuitry 264 captures images using various methods such as X-ray, ultrasound, spot examination, magnetic resonance imaging (MRI). The subcomponents of the image generator circuitry 264 (e.g., the cranial-caudal (CC) contrast enhanced mammography (CEM) generator 402, a mediolateral oblique (MLO) CEM generator 404, contrasted-enhanced (CE) digital breast tomosynthesis (DBT) generation circuitry 406, a mediolateral (ML) CEM generator 408, ultrasound generator circuitry 410, magnified image circuitry 412, a spot examination circuitry 414, and MRI generator circuitry 416, and DBT generator circuitry 418, etc.) used to generate the various breast images are further described in connection with FIG. 4.

In some examples, the image generator circuitry 264 performs a magnified image protocol (e.g., magnification view), which is a subset of the mammographic X-ray image. In such examples, the breast is placed in a specific support (e.g., a mag stand) which is approximately thirty centimeters above the detector 108. By physically moving the breast closer to the source 102, a physical magnification (e.g., zoom) is achieved. The medical imaging device 100, by activating the magnified image protocol, increases a likelihood to depict small calcifications in the breast compared to a standard mammogram. Magnification view images are typically performed at the end of a screening protocol or after a patient is recalled. In some examples, a technologist determines to perform the magnification view at the end of the screening protocol, while the radiologist, after reviewing the findings, determines to perform the magnification view after the patient is recalled.

The example timer circuitry 266 tracks elapsed units of time (e.g., minutes, hours, days, etc.). For example, some mammographic images allow for a contrast agent (e.g., iodine) to be absorbed in the tissue of the breast of the patient. In such examples, the contrast agent that is absorbed (e.g., absorbed contrast agent, observed contrast agent, etc.) is to be in the breast for longer than a first time period (e.g., longer than 5 minutes) but shorter than a second time period (e.g., shorter than 10 minutes). The timer circuitry 266 tracks when the contrast agent is injected and outputs an alert at some point between the first time and the second time. After outputting an alert to a technician, the image generator circuitry 264 configures the medical imaging device 100 to take a subsequent medical image (e.g., once the contrast agent has been in the breast for the allotted time), which may be taken after input from the technician, for example.

In some examples, the timer circuitry 266 is used to schedule future examinations. For example, the timer circuitry 266 indicates to a patient to return to the office for further examinations within a time period (e.g., one hour, one day, one week, one month, six months, one year etc.). The timer circuitry 266 calculates a waiting period before the patient is scheduled to return. In some examples, the timer circuitry 266 calculates the waiting period based on findings of the image processor circuitry 262.

The user interface circuitry 274 is used by a technologist and/or a radiologist to manually input commands (e.g., instructions) that are used by the image generator circuitry 264 to generate various images. In some examples, the user interface circuitry 274 is implemented as a touchscreen enabled display. In such examples, the display of the user interface circuitry 274, in response to a determination from the image positioning circuitry 302, indicates an alert to a user (e.g., radiologist, technologist) or an external radiology system to reposition a patient.

The local image capture protocol is stored in the local image capture protocol data store 120. The various images that are captured by the image generator circuitry 264 such as an X-ray image, an ultrasound image, a two-dimensional image, a three-dimensional image, a contrast enhanced image that includes a before-contrast image and an after-contrast image are all stored in the local patient data store 270. In addition, the local patient data store 270 includes patient demographic data. The local hospital data store 272 stores a number of medical imaging devices and a location of the medical imaging devices.

For example, the medical device circuitry 250 implemented on the first medical imaging device 100A (FIG. 1C), accesses the first image capture protocol 120A (FIG. 1C) that is stored in the local image capture protocol data store 120 with the protocol executor circuitry 258. The example updater circuitry 260 then determines if first local image capture protocol 120A (FIG. 1C) is to be updated. If an example patient has a DBT screening appointment, the protocol executor circuitry 258 loads a DBT screening protocol, which includes instructions, for example, for the medical device circuitry 250 to capture a DBT screening image and check a positioning of the DBT screening image. The DBT screening protocol could include instructions for the medical device circuitry 250 to, after checking the positioning of the DBT screening image, to either alert a technologist to adjust a positioning of the patient if the positioning is incorrect or store the captured DBT screening image if the DBT screening image is accurate.

After loading the DBT screening protocol and the patient arrives in the examination room, the example image generator circuitry 264 captures a DBT screening image (e.g., by using example DBT generator circuitry 418 of FIG. 4). If the patient is not positioned correctly which makes the DBT screening image not usable for diagnosing, the example image processor circuitry 262, after analyzing the DBT screening image, determines that the DBT screening image does not have correct positioning (e.g., by using example image positioning circuitry 302 of FIG. 3). The example user interface circuitry 274 then alerts the technologist to adjust a positioning of the patient so that a subsequent DBT screening image has correct positioning. FIG. 3 is a block diagram of an example implementation of the image processor circuitry 262 of the medical device circuitry 250 of FIG. 2. The example implementation of the image processor circuitry 262 of FIG. 3 is to analyze mammographic images. The image processor circuitry 262 of FIG. 2 may be instantiated (e.g., creating an instance of, bring into being for any length of time, materialize, implement, etc.) by programmable circuitry such as a Central Processor Unit (CPU) executing first instructions. Additionally or alternatively, the image processor circuitry 262 of FIG. 3 may be instantiated (e.g., creating an instance of, bring into being for any length of time, materialize, implement, etc.) by (i) an Application Specific Integrated Circuit (ASIC) and/or (ii) a Field Programmable Gate Array (FPGA) structured and/or configured in response to execution of second instructions to perform operations corresponding to the first instructions. It should be understood that some or all of the circuitry of FIG. 3 may, thus, be instantiated at the same or different times. Some or all of the circuitry of FIG. 3 may be instantiated, for example, in one or more threads executing concurrently on hardware and/or in series on hardware. Moreover, in some examples, some or all of the circuitry of FIG. 3 may be implemented by microprocessor circuitry executing instructions and/or FPGA circuitry performing operations to implement one or more virtual machines and/or containers.

As mentioned in connection with FIG. 2, the image processor circuitry 262 includes subcomponents such as an image positioning circuitry 302, image quality circuitry 304, findings circuitry 306, density assessment circuitry 308, risk score calculator circuitry 310, Breast-Imaging Reporting and Data System (BIRADS) score calculator circuitry 312, Background Parenchymal Enhancement (BPE) analyzer circuitry 314, image management circuitry 316, and analyzer circuitry 318.

The image positioning circuitry 302 performs an analysis to determine if a breast of a patient is aligned on the detector of the medical image device. By aligning the breast of the patient on the detector, the image that is captured is useful. For example, if the breast of the patient is not aligned on the detector, the image positioning circuitry 302 tries to compare a suggested position for the breast of the patient and an actual position of the breast of the patient. By comparing these two positions and determining that the positions are not aligned, the image positioning circuitry 302 then instructs the user interface circuitry 274 to indicate to a technician that the patient is to be readjusted so that the actual position of the breast matches the suggested positioning of the breast.

The image quality circuitry 304 checks for clarity (e.g., blurriness, sharpness etc.) of the mammographic images. If the image quality circuitry 304 determines that an image is not of a sufficient quality, the image quality circuitry 304 instructs the image generator circuitry 264 to take a subsequent replacement image. In some examples, if the image quality circuitry 304 determines that the subsequent image is also not of sufficient quality, the image quality circuitry 304 directs the user interface circuitry 274 to notify a user (e.g., technician, radiologist).

The findings circuitry 306 analyzes different findings in a breast of a patient. For example, findings include a suspicious finding or a non-suspicious finding. A first example finding may be micro-calcifications that indicate there is likely a tumor in the breast of the patient. The findings circuitry 306 determines findings by comparing different highlighted regions in the mammographic image. In some examples, the findings circuitry 306 uses the AI model inference circuitry 256 for computer-aided detection. In other examples the findings circuitry 306 uses a procedural method to determine and analyze regions in the image that stand out from the background of the image.

The density assessment circuitry 308 determines a density of a breast of a patient. For example, a breast can be classified as at least one of A density, B density, C density, or D density where breasts of A density or B density are determined to not be dense. In such examples, a breast of C density or D density is denser than a breast of A density or B density. After analyzing the density of the breast, the density assessment circuitry 308 outputs the density information. The image processor circuitry 262 uses the density determination to determine which operations to execute next in the image capture protocol. For example, if the patient has either A or B density, the density assessment circuitry 308 determines to schedule a follow-up examination some months in the future. Alternatively, if the patient has C density, the density assessment circuitry 308 determines to perform an ultrasound examination. Alternatively, if the patient has D density, the density assessment circuitry 308 determines to perform a contrast-enhanced examination.

The risk score calculator circuitry 310 calculates a risk score that may be based on findings, breast texture analysis, and breast analysis. In some examples, if the risk score is at least twenty percent (e.g., 20%+lifetime risk of breast cancer score), the risk score calculator circuitry 310 recommends an additional MRI exam for supplemental screening. For example, different images can include different findings that have been determined by the findings circuitry 306. For example, if there are numerous findings (e.g., an amount of findings over a preset threshold), then the risk score calculator circuitry 310 determines that the patient has a high risk for cancer. For example, the risk score calculator circuitry 310 determines that a patient has a high risk for cancer if there are ______ amount of findings or ______ type of findings. Alternatively, the risk score calculator circuitry 310 determines that a patient has a low risk for cancer if there are ______ amount of findings or ______ type of findings. As used herein, the risk score calculator circuitry 310 determines a risk score that is different from the BI-RADS score calculator circuitry 312 (e.g., a future breast cancer risk system).

The (Breast-Imaging Reporting and Data System) BI-RADS score calculator circuitry 312 is to determine, based on the BI-RADS factors, a level of risk of the patient. For example, the BI-RADS factors include masses in the breast (e.g., size, borders, density), breast density, calcifications, asymmetry, and tissue lesions. The BI-RADS score calculator circuitry 312 uses standard ratings of a category 0 (additional imaging required) to category 5 (high likelihood of cancer), with category 6 being proven cancer. For example, if the BI-RADS score calculator circuitry 312 determines that a patient image has a rating of category 0 (additional image required), then the BI-RADS score calculator circuitry 312 alerts the image generator circuitry 264 for a requested additional image. For example, if the BI-RADS score calculator circuitry 312 determines that a patient image has a rating of category 1 (not cancer), then the BI-RADS score calculator circuitry 312 determines that a follow-up visit is to be scheduled in a first time period (e.g., one year). However, if the BI-RADS score calculator circuitry 312 determines that a patient image has a rating of category 3 (suspected cancer), then the BI-RADS score calculator circuitry 312 determines that additional mammographic images are to be captured by the image generator circuitry 264. The seven BI-RADS scores are category 0 (e.g., additional imaging required), category 1 (e.g., no masses, calcifications, asymmetry, or other abnormalities have been found), category 2 (e.g., benign findings or noncancerous abnormalities), category 3 (e.g., findings that are probably benign, findings are unlikely to be cancerous), category 4 (e.g., suspected cancer with various percentages such as two to 10 percent, ten to fifty percent, or fifty to ninety-five percent), category 5 (e.g., highly suspected to be cancer with a percentage over ninety-five percent), and category 6 (e.g., previously determined cancer, proven cancer).

The background parenchymal enhancement (BPE) analyzer circuitry 314 analyzes the background parenchymal enhancement of the breast. In simple terms, BPE is a measurement that corresponds to an amount of absorption of contrast of the normal tissue of the breast. For example, breasts that absorb more contrast typically correspond to breasts that are at a higher risk for having cancer.

The image management circuitry 316 accesses images from the local patient data store 270 for use by the image positioning circuitry 302, the image quality circuitry 304, the findings circuitry 306, the density assessment circuitry 308, the risk score calculator circuitry 310, the BI-RADS score calculator circuitry 312, and the BPE analyzer circuitry 314. The example image management circuitry 316 determines if prior mammographic images exist and loads these prior images into the analyzer circuitry 318.

The example analyzer circuitry 318 performs image processing by using any of the image positioning circuitry 302, image quality circuitry 304, findings circuitry 306, density assessment circuitry 308, risk score calculator circuitry 310, Breast-Imaging Reporting and Data System (BIRAD) score calculator circuitry 312, Background Parenchymal Enhancement (BPE) analyzer circuitry 314, image management circuitry 316. For example, the analyzer circuitry 318 outputs recommendations based on the analysis that is performed by the image positioning circuitry 302, image quality circuitry 304, findings circuitry 306, density assessment circuitry 308, risk score calculator circuitry 310, Breast-Imaging Reporting and Data System (BIRAD) score calculator circuitry 312, and Background Parenchymal Enhancement (BPE) analyzer circuitry 314. For example, the analyzer circuitry 318 outputs a positive recommendation for additional examinations, a negative recommendation for additional examinations, a scheduling of a follow-up visit based on the analysis of the image processor circuitry 262. In some examples, the analyzer circuitry 318, in determining a recommendation for a specific follow-up examination uses patient information (e.g., pacemaker, other medical implants, etc.) and patient preference (e.g., claustrophobia in an MRI machine, travel time to hospital, etc.) to determine which specific follow-up examination to recommend. For example, if a patient does not prefer an MRI examination, then the analyzer circuitry 318 may recommend an additional X-ray or an ultrasound.

FIG. 4 is a block diagram of an example implementation of the image generator circuitry 264 of the medical device circuitry 250 of FIG. 2. The example implementation of the image generator circuitry 264 of FIG. 3 is to capture mammographic images. The image generator circuitry 264 of FIG. 2 may be instantiated (e.g., creating an instance of, bring into being for any length of time, materialize, implement, etc.) by programmable circuitry such as a Central Processor Unit (CPU) executing first instructions. Additionally or alternatively, the image generator circuitry 264 of FIG. 4 may be instantiated (e.g., creating an instance of, bring into being for any length of time, materialize, implement, etc.) by (i) an Application Specific Integrated Circuit (ASIC) and/or (ii) a Field Programmable Gate Array (FPGA) structured and/or configured in response to execution of second instructions to perform operations corresponding to the first instructions. It should be understood that some or all of the circuitry of FIG. 4 may, thus, be instantiated at the same or different times. Some or all of the circuitry of FIG. 4 may be instantiated, for example, in one or more threads executing concurrently on hardware and/or in series on hardware. Moreover, in some examples, some or all of the circuitry of FIG. 4 may be implemented by microprocessor circuitry executing instructions and/or FPGA circuitry performing operations to implement one or more virtual machines and/or containers.

The image generator circuitry 264 includes different subcomponents (e.g., protocols, software, hardware, computer architectures, etc.) that access different machinery (e.g., ultrasound wave generator, X-ray source, magnet in MRI, detector surface, etc.) of the medical device. The subcomponents of the image generator circuitry 264 include contrast enhanced mammography (CEM) generator circuitry 401 (which includes the cranial-caudal (CC) CEM generator circuitry 402, mediolateral oblique (MLO) CEM generator circuitry 404, contrasted-enhanced (CE) digital breast tomosynthesis (DBT) generation circuitry 406, mediolateral (ML) CEM generator circuitry 408), ultrasound generator circuitry 410, magnified image circuitry 412, spot examination circuitry 414, magnetic resonance imaging (MRI) generator circuitry 416, and DBT generator circuitry 418.

For example, the CEM generator circuitry 401 obtains a contrast enhanced mammography image sequence including an image that is taken after the contrast agent is injected into the arm of the patient and travels to breast tissue. In some examples, the CEM generator circuitry 401 uses dual-energy acquisition to generate the contrast enhanced mammography image sequence. By using different X-ray photon energy spectra at the same tube position, the CEM generator circuitry 401 is able to highlight different structures that have different attenuation properties at the different X-ray photon energy spectra. In some examples, the attenuation properties of the contrast agent are known. In such examples, the contrast agent may be digitally removed to clarify the underlying structures. In other examples, the contrast agent is used as a map of the patient structures. The CEM images include various viewpoints (e.g., orientations, camera locations). For example, the CC CEM generator circuitry 402 aligns an X-ray source over the breast in a cranial-caudal orientation. The MLO CEM generator circuitry 404 rotates the X-ray source to be angled over the breast to generate a mediolateral oblique view. The ML CEM generator circuitry 408 further rotates the X-ray source to be orthogonal to the CC view. The CC image, the ML image, and the MLO image are typically used as a two-dimensional representation of the three-dimensional breast. In some examples, the ML image is used to supplement the CC view and the MLO view.

The CEDBT generator circuitry 406 generates a digital breast tomographic image. A contrast enhanced digital breast tomographic image is a three-dimensional image that is generated when an X-ray source is rotated around a breast of the patient who has been injected with a contrasting agent in the arm. The example DBT generator circuitry 418 is similar to the CEDBT generator circuitry 406 but has some differences. The example DBT generator circuitry 418 is typically used for initial screening. In some examples, the DBT generator circuitry 418 is also used for capturing diagnostic data. In some examples, the DBT generator circuitry 418 generates digital breast tomographic images on patients that have not been injected with contrast, and the digital breast tomographic images are not dual energy images.

The ultrasound generator circuitry 410 generates ultrasound images by emitting an ultrasound wave and recording how the ultrasound wave interacts with tissue in a breast.

The magnified image circuitry 412 performs a magnified imaging protocol by generating a magnified image that provides more detail than initial screening images. For example, the magnified image circuitry 412, in response to an indication of a suspicious finding from the findings circuitry 306, captures a zoomed-in image of the suspicious finding. In some examples, the magnified image circuitry 412 performs a magnified image protocol (e.g., magnification view) which has the breast placed on a mag stand (e.g., 30 cm above the detector 108). By physically moving the breast closer to the source 102, a physical magnification (e.g., zoom) is achieved. The medical imaging device 100, by activating the magnified image protocol, increases a likelihood to depict small calcifications in the breast compared to a standard mammogram.

The spot examination circuitry 414 performs a spot examination protocol by activating a compression plate to slightly squeeze a breast of a patient before capturing the image. When a breast is not compressed, certain tissues can appear suspicious. By compressing the breast of the patient, different types of tissues separate which is used to distinguish between a finding (e.g., microcalcification, abnormality, masses) and a mere overlap of tissues that appears suspicious when uncompressed.

The MRI generator circuitry 416 generates a magnetic resonance image (MRI) without the usage of ionizing X-rays. An MRI is generated when the medical imaging device 100 uses a large magnet to generate a magnetic field around a patient. After generating the magnetic field, the medical imaging device 100 then sends pulses of radio waves. Due to the magnetic field, atoms in the body of the patient align to a first direction, and are moved out of the first direction by the pulses of radio waves. After the medical imaging device 100 turns off the pulses of radio waves, the atoms revert back to the first direction based on the magnetic field. The MRI generator circuitry 416 then detects energy released as the atoms revert back to the first direction to generate a 3D breast image. The X-ray generator circuitry 420 generates X-ray images (e.g., computed tomography (CT) images). An X-ray is a form of electromagnetic radiation that passes through tissues of objects (e.g., a human body). The X-ray beam passes through the objects and a detector 108 (FIG. 1A), while certain portions of the X-ray beam are blocked by other structures in the body (e.g., bones). The detector 108 (FIG. 1A) then generates the X-ray image based on interactions of the X-ray beam and the objects. Tumors tend to appear as masses that are brighter than background of the generated X-ray image.

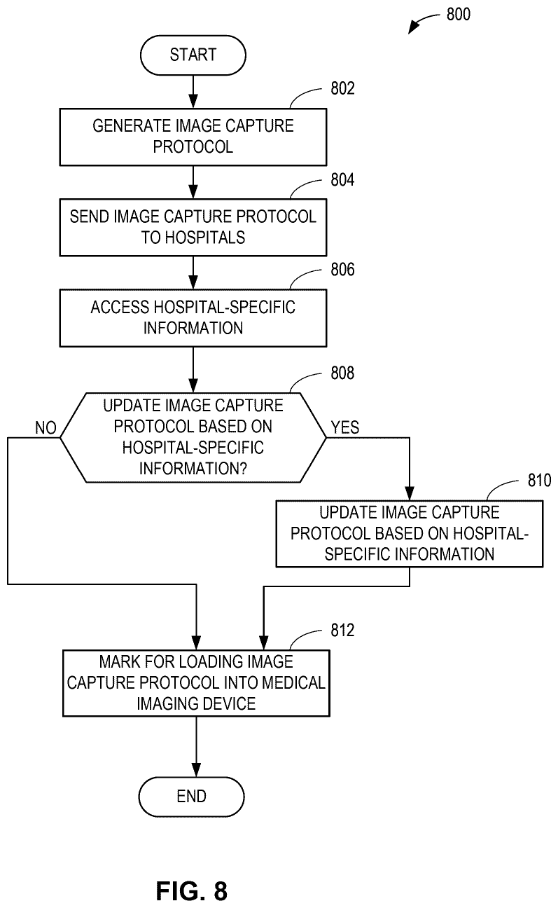

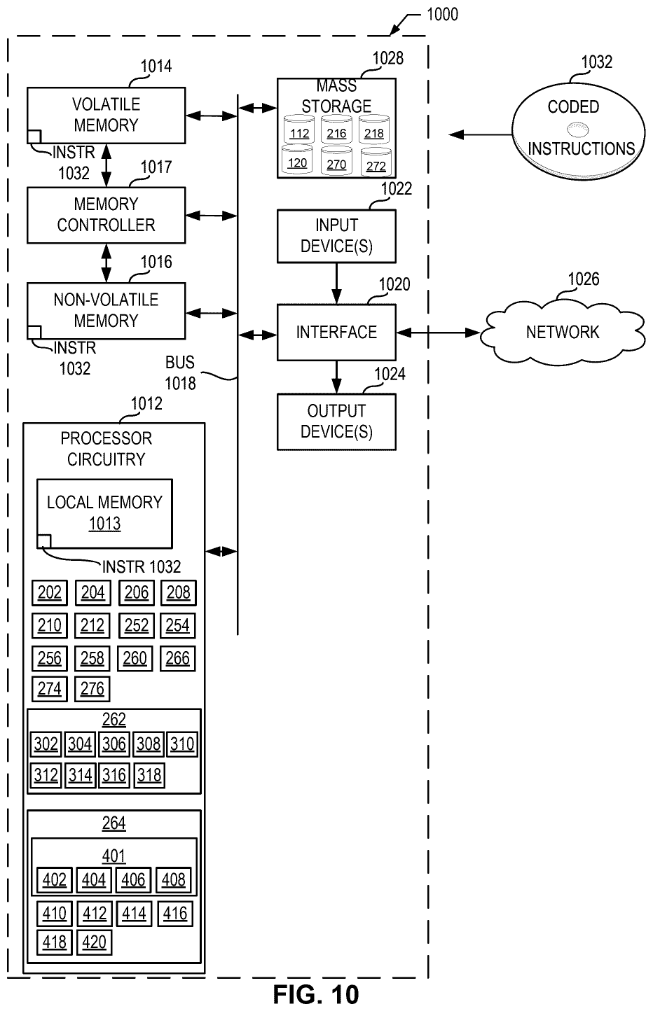

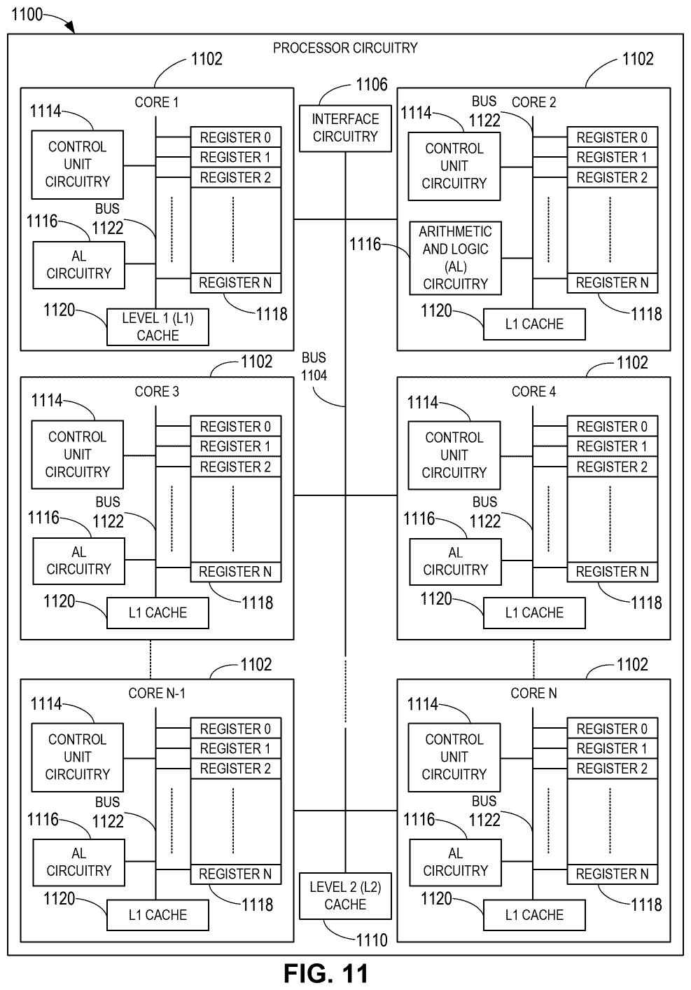

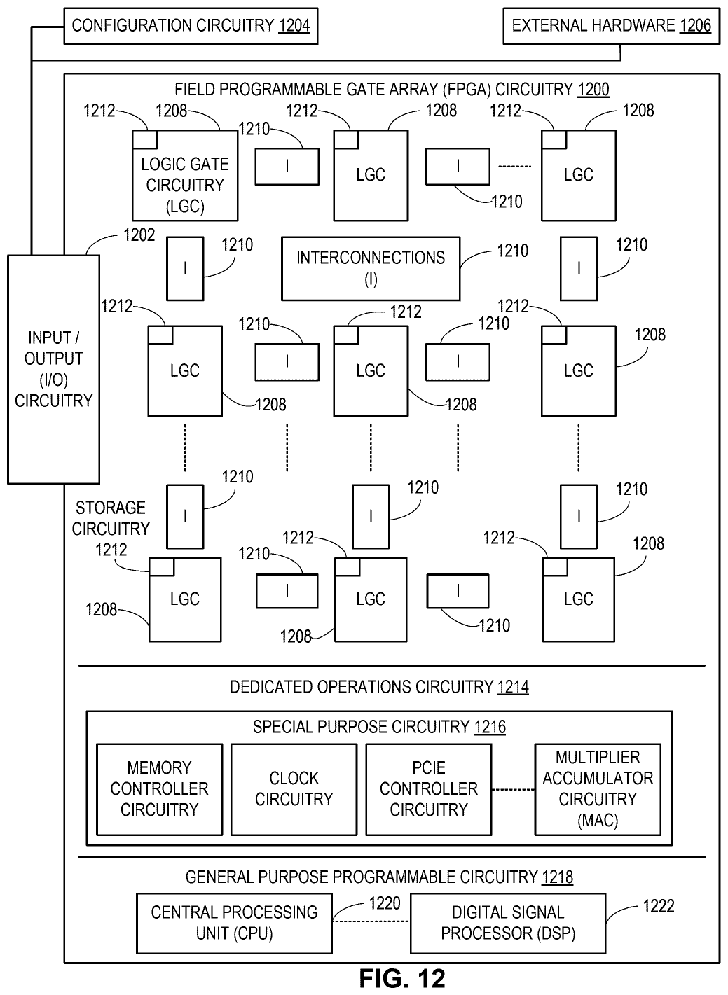

In some examples, the breast image processing circuitry 110 includes means for transmitting patient data, image protocol information, and hospital data which may be implemented by the network interface 202. For instance, the network interface 202 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least blocks 804 and 806 of FIG. 8.

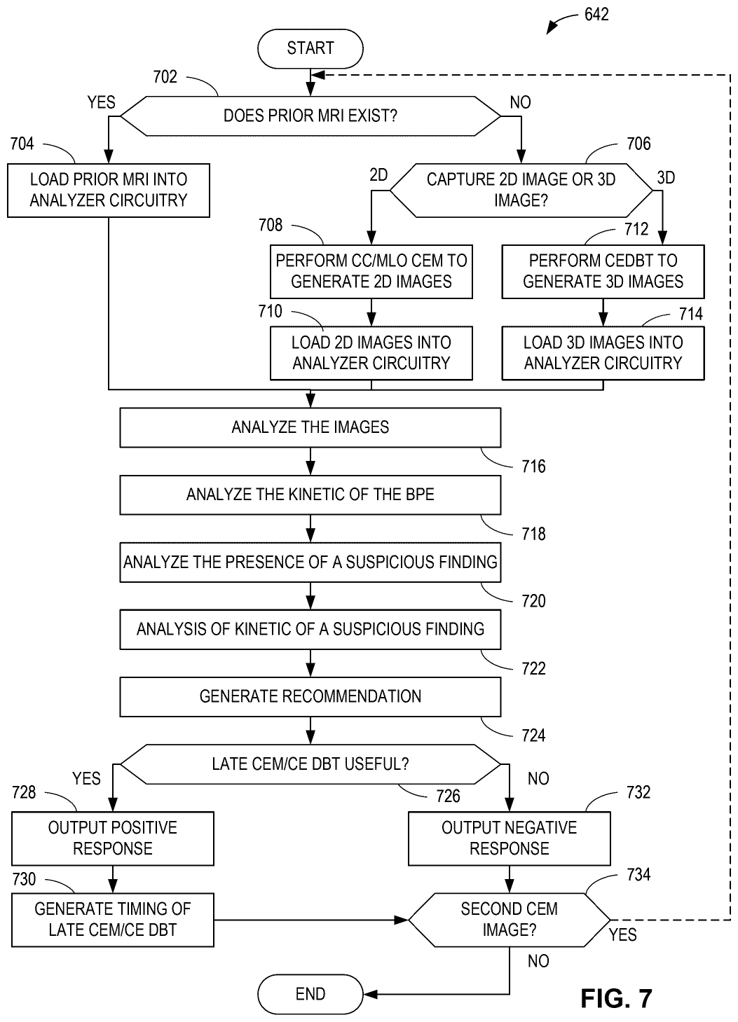

In some examples, the breast image processing circuitry 110 includes means for training an artificial intelligence model which may be implemented by the AI model training circuitry 204. For instance, the AI model training circuitry 204 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least blocks 728 and 732 of FIG. 7.

In some examples, the breast image processing circuitry 110 includes means for training an artificial intelligence model which may be implemented by the AI model training circuitry 204. For instance, the AI model training circuitry 204 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least blocks 728 and 732 of FIG. 7.

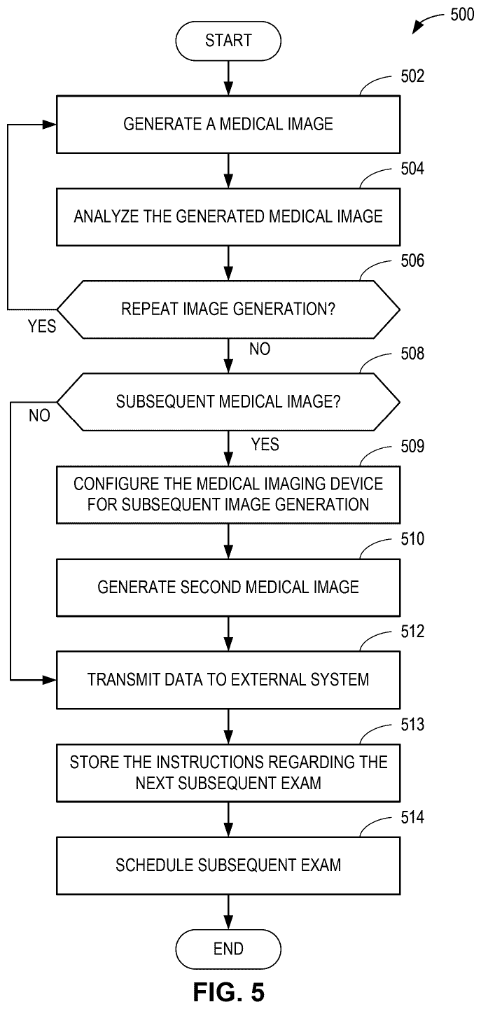

In some examples, the breast image processing circuitry 110 includes means for performing inference with an artificial intelligence model which may be implemented by the AI model inference circuitry 206. For instance, the AI model inference circuitry 206 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least block 504 of FIG. 5 and blocks 716, 718, 720, 722, 724 of FIG. 7.

In some examples, the breast image processing circuitry 110 includes means for generating an image capture protocol which may be implemented by the image capture circuitry 208. For instance, the image capture circuitry 208 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least block 802 of FIG. 8.

In some examples, the breast image processing circuitry 110 includes means for updating an image capture protocol which may be implemented by the updater circuitry 210. For instance, the updater circuitry 210 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least blocks 808, 810, and 812 of FIG. 8.

In some examples, the breast image processing circuitry 110 includes means for performing image processing which may be implemented by the image processor circuitry 212. For instance, the image processor circuitry 212 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least blocks 504, 506, and 508 of FIG. 5.

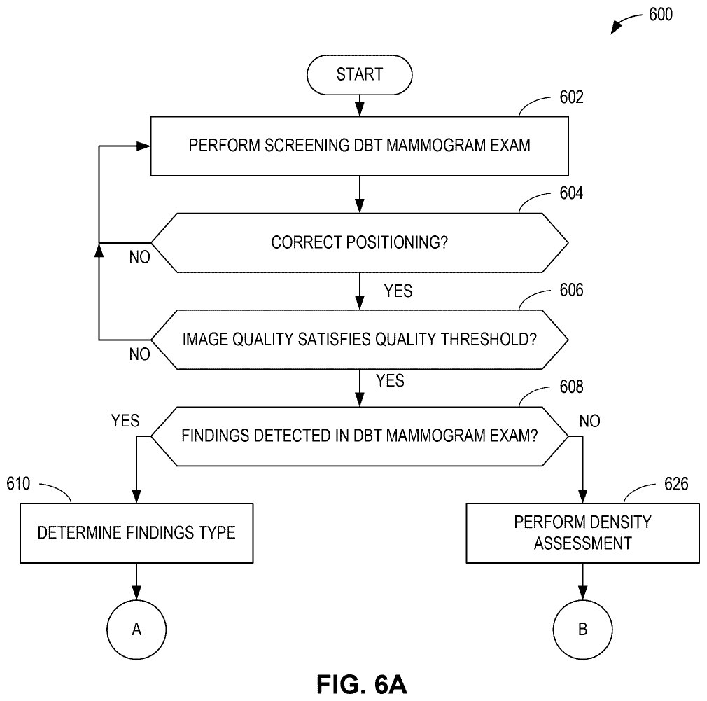

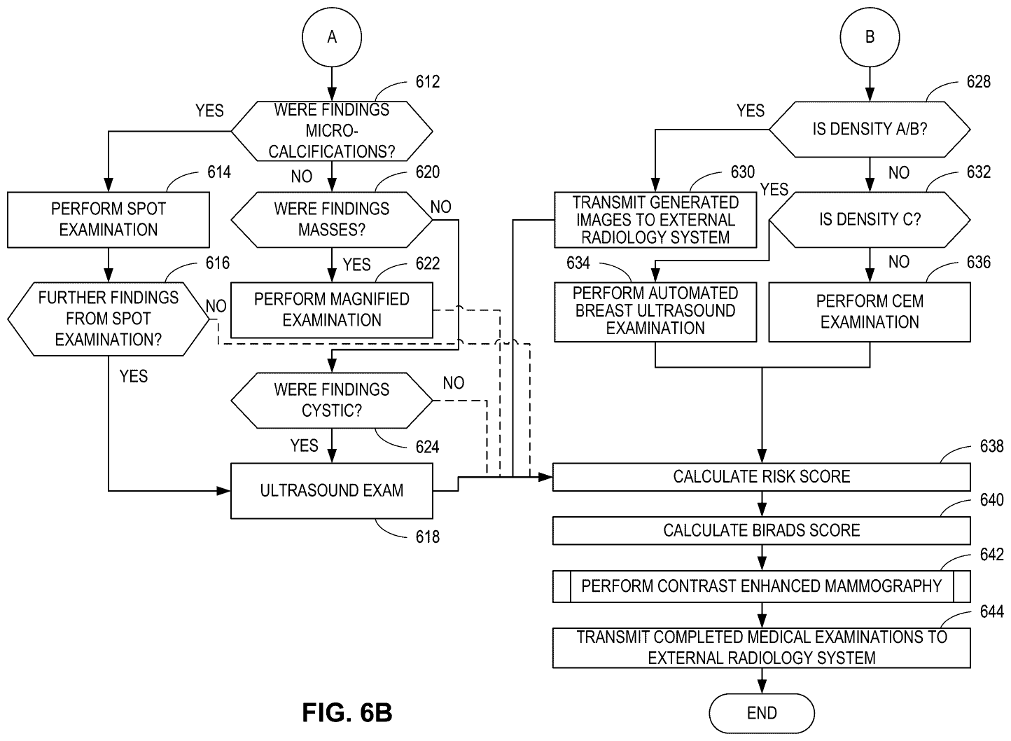

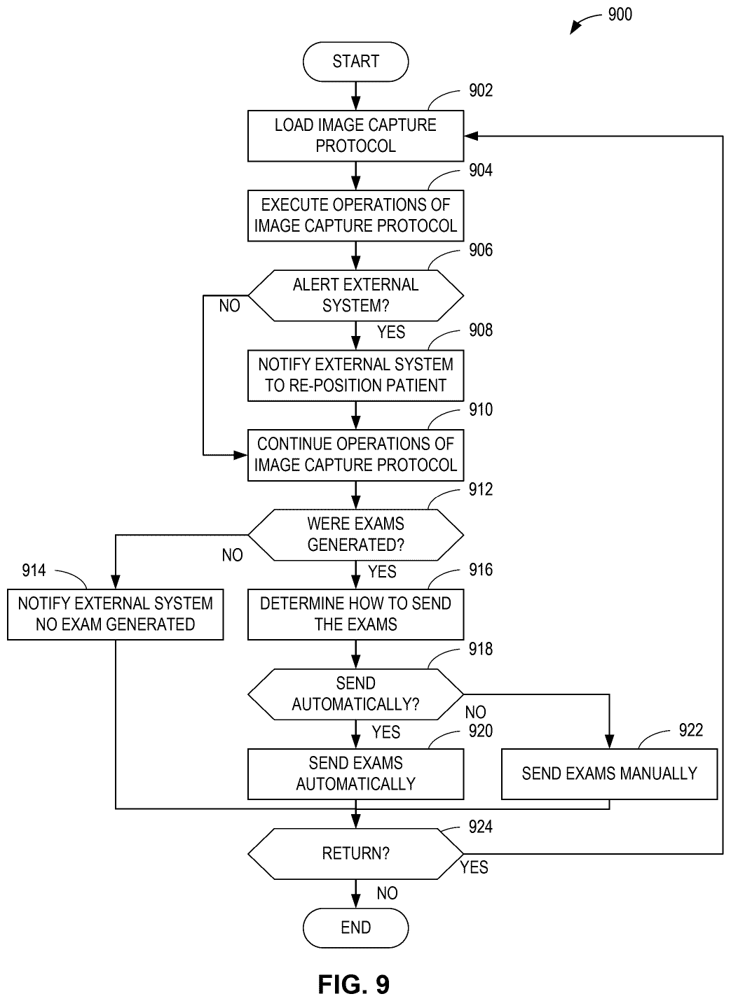

In some examples, the medical device circuitry 250 includes means for transmitting patient data, image protocol information, and hospital data which may be implemented by the network interface 252. For instance, the network interface 252 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least blocks 512, 514, of FIG. 5, blocks 630, 644 of FIG. 6 and block 920 of FIG. 9.

In some examples, the medical device circuitry 250 includes means for training an artificial intelligence model which may be implemented by the AI model training circuitry 254. For instance, the AI model training circuitry 254 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least blocks 728 and 732 of FIG. 7.

In some examples, the medical device circuitry 250 includes means for performing inference with an artificial intelligence model which may be implemented by the AI model inference circuitry 256. For instance, the AI model inference circuitry 256 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least block 504 of FIG. 5 and blocks 716, 718, 720, 722, 724 of FIG. 7.

In some examples, the medical device circuitry 250 includes means for executing operations of an image capture protocol which may be implemented by the protocol executor circuitry 258. For instance, the protocol executor circuitry 258 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least block 508 of FIG. 5 and blocks 902, 912, 916, 918, and 924 of FIG. 9.

In some examples, the medical device circuitry 250 includes means for updating an image capture protocol which may be implemented by the updater circuitry 260. For instance, the updater circuitry 260 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least blocks 808, 810, and 812 of FIG. 8.

In some examples, the medical device circuitry 250 includes means for processing images which may be implemented by the image processor circuitry 262. For instance, the image processor circuitry 262 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least blocks 504, 506, 508, 513 of FIG. 5, blocks 808, 810, and 812 of FIG. 8 (and associated blocks 604, 606, 608, 610, 626 of FIG. 6A, blocks 612, 620, 616, 624, 628, 632,638, 640 of FIG. 6B, blocks 702, 704, 706, 710, 714, 716, 718, 720, 722, 724,726, 728, 732, 734 of FIG. 7).

In some examples, the medical device circuitry 250 includes means for generating images which may be implemented by the image generator circuitry 264. For instance, the image generator circuitry 264 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least blocks 502, 509 and 510 of FIG. 5 (and associated blocks 602 of FIG. 6A, blocks 614, 622, 618, 634, 636, 642 of FIG. 6B, blocks 708, 712 of FIG. 7).

In some examples, the medical device circuitry 250 includes means for tracking a passage of time which may be implemented by the timer circuitry 266. For instance, the timer circuitry 266 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least block 630 of FIG. 6B and block 730 of FIG. 7.

In some examples, the medical device circuitry 250 includes means for allowing a user to interact with the medical device which may be implemented by the user interface circuitry 274. For instance, the user interface circuitry 274 may be instantiated by the example programmable circuitry 1012 of FIG. 10, the example microprocessor 1100 of FIG. 11, or the FPGA circuitry 1200 of FIG. 12 executing machine executable instructions or operations corresponding to the machine readable instructions such as those implemented by at least blocks 514 of FIG. 5, and blocks 906, 908, 914, 922, of FIG. 9.

While an example manner of implementing the medical device circuitry 250 and the breast imaging processing circuitry 110 are illustrated in FIG. 2, one or more of the elements, processes, and/or devices illustrated in FIG. 2 may be combined, divided, re-arranged, omitted, eliminated, and/or implemented in any other way. Further, the example network interface 202, example AI model training circuitry 204, example AI model inference circuitry 206, example image capture circuitry 208, example updater circuitry 210, example image processor circuitry 212, the example network interface 252, example AI model training circuitry 254, example AI model inference circuitry 256, example protocol executor circuitry 258, example updater circuitry 260, example image processor circuitry 262, example image generator circuitry 264, example timer circuitry 266, and/or, more generally, the example medical device circuitry 250 of FIG. 2 or the breast imaging processing circuitry 110 of FIG. 2, may be implemented by hardware alone or by hardware in combination with software and/or firmware. Thus, for example, any of the example network interface 202, example AI model training circuitry 204, example AI model inference circuitry 206, example image capture circuitry 208, example updater circuitry 210, example image processor circuitry 212, the example network interface 252, example AI model training circuitry 254, example AI model inference circuitry 256, example protocol executor circuitry 258, example updater circuitry 260, example image processor circuitry 262, example image generator circuitry 264, example timer circuitry 266, and/or, more generally, the example medical device circuitry 250, could be implemented by programmable circuitry in combination with machine readable instructions (e.g., firmware or software), processor circuitry, analog circuit(s), digital circuit(s), logic circuit(s), programmable processor(s), programmable microcontroller(s), graphics processing unit(s) (GPU(s)), digital signal processor(s) (DSP(s)), ASIC(s), programmable logic device(s) (PLD(s)), and/or field programmable logic device(s) (FPLD(s)) such as FPGAs. Further still, the example medical device circuitry 250 of FIG. 2 and the breast imaging processing circuitry 110 of FIG. 2 may include one or more elements, processes, and/or devices in addition to, or instead of, those illustrated in FIG. 2, and/or may include more than one of any or all of the illustrated elements, processes and devices.

Flowchart(s) representative of example machine readable instructions, which may be executed by programmable circuitry to implement and/or instantiate the medical device circuitry 250 of FIG. 2 and/or the breast imaging processing circuitry 110 of FIG. 2 and/or representative of example operations which may be performed by programmable circuitry to implement and/or instantiate the medical device circuitry 250 of FIG. 2 and/or the breast imaging processing circuitry 110 of FIG. 2, are shown in FIGS. 5-9. The machine readable instructions may be one or more executable programs or portion(s) of one or more executable programs for execution by programmable circuitry such as the programmable circuitry 1012 shown in the example programmable circuitry platform 1000 discussed below in connection with FIG. 10 and/or may be one or more function(s) or portion(s) of functions to be performed by the example programmable circuitry (e.g., an FPGA) discussed below in connection with FIGS. 11 and/or 12. In some examples, the machine readable instructions cause an operation, a task, etc., to be carried out and/or performed in an automated manner in the real world. As used herein, “automated” means without human involvement.

The program may be embodied in instructions (e.g., software and/or firmware) stored on one or more non-transitory computer readable and/or machine readable storage medium such as cache memory, a magnetic-storage device or disk (e.g., a floppy disk, a Hard Disk Drive (HDD), etc.), an optical-storage device or disk (e.g., a Blu-ray disk, a Compact Disk (CD), a Digital Versatile Disk (DVD), etc.), a Redundant Array of Independent Disks (RAID), a register, ROM, a solid-state drive (SSD), SSD memory, non-volatile memory (e.g., electrically erasable programmable read-only memory (EEPROM), flash memory, etc.), volatile memory (e.g., Random Access Memory (RAM) of any type, etc.), and/or any other storage device or storage disk. The instructions of the non-transitory computer readable and/or machine readable medium may program and/or be executed by programmable circuitry located in one or more hardware devices, but the entire program and/or parts thereof could alternatively be executed and/or instantiated by one or more hardware devices other than the programmable circuitry and/or embodied in dedicated hardware. The machine readable instructions may be distributed across multiple hardware devices and/or executed by two or more hardware devices (e.g., a server and a client hardware device). For example, the client hardware device may be implemented by an endpoint client hardware device (e.g., a hardware device associated with a human and/or machine user) or an intermediate client hardware device gateway (e.g., a radio access network (RAN)) that may facilitate communication between a server and an endpoint client hardware device. Similarly, the non-transitory computer readable storage medium may include one or more mediums. Further, although the example program is described with reference to the flowchart(s) illustrated in FIGS. 5-9, many other methods of implementing the example medical device circuitry 250 and/or the breast imaging processing circuitry 110 of FIG. 2 may alternatively be used. For example, the order of execution of the blocks of the flowchart(s) may be changed, and/or some of the blocks described may be changed, eliminated, or combined. Additionally or alternatively, any or all of the blocks of the flow chart may be implemented by one or more hardware circuits (e.g., processor circuitry, discrete and/or integrated analog and/or digital circuitry, an FPGA, an ASIC, a comparator, an operational-amplifier (op-amp), a logic circuit, etc.) structured to perform the corresponding operation without executing software or firmware. The programmable circuitry may be distributed in different network locations and/or local to one or more hardware devices (e.g., a single-core processor (e.g., a single core CPU), a multi-core processor (e.g., a multi-core CPU, an XPU, etc.)). For example, the programmable circuitry may be a CPU and/or an FPGA located in the same package (e.g., the same integrated circuit (IC) package or in two or more separate housings), one or more processors in a single machine, multiple processors distributed across multiple servers of a server rack, multiple processors distributed across one or more server racks, etc., and/or any combination(s) thereof.

The machine readable instructions described herein may be stored in one or more of a compressed format, an encrypted format, a fragmented format, a compiled format, an executable format, a packaged format, etc. Machine readable instructions as described herein may be stored as data (e.g., computer-readable data, machine-readable data, one or more bits (e.g., one or more computer-readable bits, one or more machine-readable bits, etc.), a bitstream (e.g., a computer-readable bitstream, a machine-readable bitstream, etc.), etc.) or a data structure (e.g., as portion(s) of instructions, code, representations of code, etc.) that may be utilized to create, manufacture, and/or produce machine executable instructions. For example, the machine readable instructions may be fragmented and stored on one or more storage devices, disks and/or computing devices (e.g., servers) located at the same or different locations of a network or collection of networks (e.g., in the cloud, in edge devices, etc.). The machine readable instructions may require one or more of installation, modification, adaptation, updating, combining, supplementing, configuring, decryption, decompression, unpacking, distribution, reassignment, compilation, etc., in order to make them directly readable, interpretable, and/or executable by a computing device and/or other machine. For example, the machine readable instructions may be stored in multiple parts, which are individually compressed, encrypted, and/or stored on separate computing devices, wherein the parts when decrypted, decompressed, and/or combined form a set of computer-executable and/or machine executable instructions that implement one or more functions and/or operations that may together form a program such as that described herein.

In another example, the machine readable instructions may be stored in a state in which they may be read by programmable circuitry, but require addition of a library (e.g., a dynamic link library (DLL)), a software development kit (SDK), an application programming interface (API), etc., in order to execute the machine-readable instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, machine readable, computer readable and/or machine readable media, as used herein, may include instructions and/or program(s) regardless of the particular format or state of the machine readable instructions and/or program(s).

The machine readable instructions described herein can be represented by any past, present, or future instruction language, scripting language, programming language, etc. For example, the machine readable instructions may be represented using any of the following languages: C, C++, Java, C#, Perl, Python, JavaScript, HyperText Markup Language (HTML), Structured Query Language (SQL), Swift, etc.

As mentioned above, the example operations of FIGS. 5-9 may be implemented using executable instructions (e.g., computer readable and/or machine readable instructions) stored on one or more non-transitory computer readable and/or machine readable media. As used herein, the terms non-transitory computer readable medium, non-transitory computer readable storage medium, non-transitory machine readable medium, and/or non-transitory machine readable storage medium are expressly defined to include any type of computer readable storage device and/or storage disk and to exclude propagating signals and to exclude transmission media. Examples of such non-transitory computer readable medium, non-transitory computer readable storage medium, non-transitory machine readable medium, and/or non-transitory machine readable storage medium include optical storage devices, magnetic storage devices, an HDD, a flash memory, a read-only memory (ROM), a CD, a DVD, a cache, a RAM of any type, a register, and/or any other storage device or storage disk in which information is stored for any duration (e.g., for extended time periods, permanently, for brief instances, for temporarily buffering, and/or for caching of the information). As used herein, the terms “non-transitory computer readable storage device” and “non-transitory machine readable storage device” are defined to include any physical (mechanical, magnetic and/or electrical) hardware to retain information for a time period, but to exclude propagating signals and to exclude transmission media. Examples of non-transitory computer readable storage devices and/or non-transitory machine readable storage devices include random access memory of any type, read only memory of any type, solid state memory, flash memory, optical discs, magnetic disks, disk drives, and/or redundant array of independent disks (RAID) systems. As used herein, the term “device” refers to physical structure such as mechanical and/or electrical equipment, hardware, and/or circuitry that may or may not be configured by computer readable instructions, machine readable instructions, etc., and/or manufactured to execute computer-readable instructions, machine-readable instructions, etc.

FIG. 5 is a flowchart representative of example machine readable instructions and/or example operations 500 that may be executed, instantiated, and/or performed by example programmable circuitry to implement the medical device circuitry 250 of FIG. 2 to determine when to generate, analyze, and transmit medical images. The example machine-readable instructions and/or the example operations 500 of FIG. 5 begin at block 502, at which the example image generator circuitry 264 generates a medical image. For example, the medical image may be generated by using at least one of the CC CEM generator circuitry 402, the MLO CEM generator circuitry 404, the CEDBT generator circuitry 406, the ML CEM generator circuitry 408, the ultrasound generator circuitry 410, the magnified image circuitry 412, the spot examination circuitry 414, and/or the MRI generator circuitry 416.

At block 504, the image processor circuitry 262 analyzes the generated medical image. In some examples, the AI model inference circuitry 206 of the breast imaging processing circuitry 110 analyzes the generated medical images. In other examples, the AI model inference circuitry 256 of the medical device circuitry 250 analyzes the generated medical images. In yet other examples, the breast imaging analysis circuitry 124 (FIG. 1D) analyzes the generates medical images.

At block 506, the image processor circuitry 262 determines to repeat image generation. For example, in response to determining to repeat generation (e.g., “YES”), control returns to block 502 where the image generator circuitry 264 generates a medical image. Alternatively, in response to determining not to repeat image generation (e.g., “NO”), control advances to block 508. The image processor circuitry 262 may determine to repeat image generation based on the image positioning circuitry 302 determining that a position was incorrect and/or the image quality circuitry 304 determining that the image is unusable.

At block 508, the image processor circuitry 262 determines to generate a subsequent medical image. For example, in response to determining to generate a subsequent medical image (e.g., “YES”), control advances to block 509. Alternatively, in response to determining not to generate a subsequent medical image (e.g., “NO”), control advances to block 512.

The example image processor circuitry 262 may determine to generate a subsequent medical image by receiving an indication from the example findings circuitry 306 determining that based on a finding in a first medical image that a second image is warranted (e.g., different view, adjusted position, different wavelength, different imaging technique etc.). In some examples, the medical device circuitry 250 transmits the generated medical image to be analyzed by the breast imaging processing circuitry 110 (FIG. 1C) and/or the breast imaging analysis circuitry 124 (FIG. 1D) and receives an instruction from the breast imaging processing circuitry 110 (FIG. 1C) and/or the breast imaging analysis circuitry 124 (FIG. 1D) and/or the breast imaging protocol circuitry 126 (FIG. 1D) to configure the medical imaging device 100 for subsequent image generation, to perform subsequent image generation, or to skip subsequent image generation. In other examples, instructions regarding the next operation are stored. In yet other examples, the medical device circuitry 250 transmits the generated medical image to be analyzed by the breast imaging processing circuitry 110 and receives an instruction to not perform repeat image generation as a separate medical imaging device (such as the example second medical imaging device 100B of FIG. 1C) is assigned to perform the subsequent image generation. For example, the first medical imaging device 100A (FIG. 1C) which implements a first instance of the medical device circuitry 250 is an ultrasound machine (e.g., ultrasound system), and the second medical imaging device 100B of FIG. 1C is an X-ray machine. The first medical imaging device 100A (FIG. 1C) generates a first type of image (e.g., ultrasound images) and the second medical imaging device 100B (FIG. 1C) generates a second type of image (e.g., X-ray images). Further details regarding determining which images and types of images to generate are described in connection with FIGS. 6A-6B.

At block 509, the image generator circuitry 264 configures the medical imaging device for subsequent image generation. For example, certain medical images require supervision and/or intervention of a technician. In such examples, the image generator circuitry 264 configures the medical imaging device 100 for the next image or next set of examinations, and awaits a confirmation from the technician. Control advances to block 510.

At block 510, the image generator circuitry 264 generates the second medical image. The second medical image may be either of the same type of medical image as the first medical image (e.g., the previous mammographic image) or a different type of image. By generating the second medical image, the techniques disclosed herein enable interaction and configuration of one or more devices with automated analysis without involvement of a radiologist. Control advances to block 512.

At block 512, the network interface 252 transmits the medical image (or images) to a computer that is operated by a radiologist (e.g., external radiology system). In some examples, the network interface 252 transmits the medical image (or images) to the breast imaging processing circuitry 110 (FIG. 1C), or the breast imaging protocol circuitry 126 (FIG. 1D). In other examples, the network interface 252 transmits the medical image (or images) to breast imaging protocol circuitry 126 (FIG. 1D) and/or the breast imaging analysis circuitry 124 (FIG. 1D).

At block 513, the image processor circuitry 262 stores the instructions regarding the next subsequent examination in the local patient data store 270 for the current patient. For example, the image processor circuitry 262 may store the instructions that if the first examination is an X-ray examination (e.g., that may include multiple X-ray images), the second examination is an ultrasound examination (e.g., that may include multiple ultrasound images). In such examples, the image processor circuitry 262, at a later time, or on a different machine may access the instructions regarding the next subsequent exam.

At block 514, the example analyzer circuitry 318 is used to schedule a subsequent exam by accessing the saved instructions from the local patient data store 270. In some examples the analysis of the generated material may be performed by AI model inference circuitry 256. In some examples, a radiologist or an external radiology system may determine when to schedule a subsequent visit based on a presence or lack of findings in the medical images. In some examples, the breast imaging protocol circuitry 126 determines when to schedule the subsequent visit. In some examples, the subsequent visit is to occur immediately in a separate examination room of the hospital (e.g., that a contrast enhanced mammographic image is to be captured). In some examples, the subsequent visit is to occur at a later time (e.g., a week, a month, a year, etc.). After block 514, the instructions 500 end.

FIG. 6 is a flowchart representative of example machine readable instructions and/or example operations 600 that may be executed, instantiated, and/or performed by programmable circuitry to generate medical images without interaction from a radiologist. FIG. 6 is split into two figures: FIG. 6A and FIG. 6B. FIG. 6A includes blocks 602, 604, 606, 608, 610, and 626 and FIG. 6B includes blocks 612, 614, 616, 618, 620, 622, 624, 628, 630, 632, 636, 638, 640, 642 and 644. Block 610 of FIG. 6A leads to block 612 of FIG. 6B. Block 626 of FIG. 6A leads to block 628 of FIG. 6B.

FIG. 6A is a first portion of a flowchart representative of example machine readable instructions and/or example operations that may be executed, instantiated, and/or performed by example programmable circuitry to implement the medical device circuitry 250 of FIG. 2. In some examples, portions of the image analysis are performed by the breast imaging processing circuitry 110 (FIG. 1C). In other examples, portions of the image analysis are performed by the breast imaging analysis circuitry 124 (FIG. 1D) and decisions regarding the imaging modality are performed by the breast imaging protocol circuitry 126 (FIG. 1D). FIG. 6A begins at block 602 where the example DBT generator circuitry 418 performs a DBT mammogram exam (e.g., a DBT screening).

At block 604, the image positioning circuitry 302 determines if the breasts of the patient are positioned correctly on a surface of the detector 108 (FIG. 1A). For example, in response to determining that the positioning is incorrect (e.g., “NO”), control returns to block 602. Alternatively, in response to determining that the positioning was correct (e.g., “YES”), control advances to block 606. The image positioning circuitry 302 may determine that the breasts are positioned correctly based on analysis of at least one of symmetry, nipple position, pectoral muscle line presence, position, etc.

At block 606, the image quality circuitry 304 determines if the image quality of the screening examination satisfies a quality threshold. For example, in response to the image quality circuitry 304 determining that the image quality satisfies a quality threshold (e.g., “YES”), control advances to block 608. Alternatively, in response to the image quality circuitry 304 determining the image quality does not satisfy a quality threshold (e.g., “NO”), control returns to block 602.

At block 608, the example findings circuitry 306 determines if findings were detected in the DBT mammogram exam (e.g., screening exam). For example, in response to determining that findings were detected in the DBT mammogram exam (e.g., “YES”), control advances to block 610. Alternatively, in response to the findings circuitry 306 determining that findings were not detected in the DBT mammogram exam (e.g., “NO”), control advances to block 626.

FIG. 6B is a second portion of a flowchart representative of example machine readable instructions and/or example operations that may be executed, instantiated, and/or performed by example programmable circuitry to implement the medical device circuitry 250 of FIG. 2. FIG. 6B continues from FIG. 6A. Continuing from block 610, at block 612, the findings circuitry 306 determines if the findings were microcalcifications. For example, in response to the findings circuitry 306 determining that the findings were microcalcifications (e.g., “YES”), control advances to block 614. Alternatively, in response to the findings circuitry 306 determining that the findings were not microcalcifications (e.g., “NO”), control advances to block 620. The example findings circuitry 306 may determine that the findings are microcalcifications by comparing the images taken by the detector with an image data store. In some examples, the findings circuitry 306 determines that the findings are microcalcifications by using the AI model inference circuitry 256.

At block 614, the spot examination circuitry 414 of the image generator circuitry 264 performs a spot examination. After the spot examination circuitry 414 performs a spot examination, control advances to block 616.

At block 616, the findings circuitry 306 determines if there were further findings from the spot examination. For example, if the findings circuitry 306 determines that there are further findings from the spot examination (e.g., “YES”), control advances to block 618. Alternatively, if the findings circuitry 306 determines that there are no further findings from the spot examination (e.g., “NO”), control advances to block 638. For example, if there is a spot examination, there is a spot in the breast to examine, which indicates that there is a finding. In some examples, an AI model implemented by on the findings circuitry 306 determines a malignancy score or a risk score based on the presence of the spot.

At block 620, regarding the findings from block 612 that were not microcalcifications, the findings circuitry 306 determines if these findings were masses. For example, in response to the findings circuitry 306 determining that the findings were masses (e.g., “YES”), control advances to block 622. Alternatively, in response to the findings circuitry 306 determining that the findings were not masses (e.g., “NO”), control advances to block 624. The example findings circuitry 306 determines that the findings were masses based on a comparison of a color of the findings (e.g., a white growth is likely a mass).

At block 622, regarding the findings from block 620 that were determined to be masses, the magnified image circuitry 412 performs a magnified image examination. For example, the magnified image circuitry 412 automatically selects “MAG VIEW” as an option on the medical imaging device 100. Once in the “MAG VIEW” option, the magnified image circuitry 412 changes the focal spot, and sends an instruction to the user interface circuitry 274 to display a photo of the mag stand (e.g., breast support) and the breast paddle to be used to guide the technician in positioning the breast for imaging. After the magnified image circuitry 412 performs the magnified image examination, control advances to block 638.

At block 624, regarding the findings from block 620 that were not determined to be masses, the findings circuitry 306 determines if the findings were cystic. For example, if the example findings circuitry 306 determines that the findings were cystic (e.g., “YES”), control advances to block 618. Alternatively, if the example findings circuitry 306 determines that the findings were not cystic (e.g., “NO”), control advances to block 638. For example, the findings circuitry 306 determines that the findings are cystic based on not being microcalcifications or masses. In some examples, the findings circuitry 306 uses a combination of multiple information sources to determine the differences between cystic findings, mass findings, and microcalcification findings. For example, the multiple informational sources include BI-RADS descriptor of the finding (e.g., shape, size, margin, etc.) and molecular composition of the finding (e.g., dual-energy). In some examples, the findings circuitry 306 confirms the type of the finding based on an ultrasound or elastography analysis. In some examples, the findings circuitry 306 differentiates between cystic, microcalcifications, and masses with a patch-based deep learning classification technique. In some examples, the findings circuitry 306 differentiates between cyst and solid mass.

At block 618, the ultrasound generator circuitry 410 performs an ultrasound. For example, the ultrasound generator circuitry 410 emits ultrasound waves that pass through the body of the patient, and after interacting with structures in the body of the patient are reflected back to the ultrasound generator circuitry 410. By emitting and reflecting ultrasound waves, the ultrasound generator circuitry 410 generates a 2D ultrasound image. After the ultrasound is performed, control advances to block 638.

At block 628, after block 626 from FIG. 6A, the density assessment circuitry 308 performs a density assessment. For example, if the density assessment circuitry 308 determines that the density is either A density or B density (e.g., “YES”), control advances to block 630. Alternatively, if the density assessment circuitry 308 determines that the density is not either A density or B density (e.g., “NO”), control advances to block 632. The example density assessment circuitry 308 may determine the density of the breast by using AI model inference circuitry 256.

At block 630, the example network interface 252 transmits the completed screening DBT mammogram examination to an external radiology system. For example, based on the A density or the B density, the medical device circuitry 250 determines that no follow-up examinations are warranted, and transmits the completed screening DBT mammogram examination for analysis by either the external radiology system or a radiologist who has access to the external radiology system. After block 630, control advances to block 638.

At block 632, regarding the densities that were not either A density or B density, the density assessment circuitry 308 determines if the density is C density. For example, if the density assessment circuitry 308 determines the density is density C (e.g., “YES”), control advances to block 634. Alternatively, if the density assessment circuitry 308 determines that the density is not density C (e.g., “NO”), control advances to block 636. By determining that the density is not A, B, or C, the density assessment circuitry 308 implicitly determines that the density is density D. However, in some examples, the density assessment circuitry 308 explicitly determines if the density is density D, and in response to not determining the density as one of A, B, C, or D, the density assessment circuitry 308 determines that the density is indeterminate.

At block 634, the ultrasound generator circuitry 410 performs an ABUS ultrasound examination. An example ABUS ultrasound (e.g., automated whole-breast ultrasound) is typically used on patients with dense breasts, saline breast implants, and silicone breast implants. Control advances to block 638.

At block 636, the example contrast-enhanced-mammography generator circuitry 401 performs the contrast enhanced mammography examination. The contrast enhanced mammography generator circuitry 401 includes the CC CEM generator circuitry 402, the MLO CEM generator circuitry 404, the ML CEM generator circuitry 408, and the CEDBT generator circuitry 406. After block 636, control advances to block 638.

At block 638, the risk score calculator circuitry 310 calculates the risk score. The example risk score calculator calculates the risk score based on a combination of multiple informational sources that include, for example, patient data, hospital data, imaging data, prior examinations, and family data. In some examples, the risk score calculator 310, by using an AI model, provides a score of likelihood of belonging to the ones of the BI-RADS classes (e.g., category 0, category 1, category 4, etc.) and takes the higher score as a final result. After calculating the risk score, control advances to block 640.

At block 640, the BI-RADS score calculator circuitry 312 calculates the BI-RADS score. In some examples, the BI-RADS score calculator circuitry 312 generates the BI-RADS score based on a deep learning technique which uses computer-aided detection on an image to assign a malignancy probability to an object/finding in the image. In some examples, the BI-RADS score calculator circuitry 312 performs analysis of characteristics of any findings. Some example characteristics include shape, contrast, heterogeneity, symmetry, and contour. After determining the characteristics of findings, the BI-RADS score calculator circuitry 312 uses a BI-RADS Atlas to associate the characteristics to a score. After block 640, control advances to block 642.

At block 642, the contrast enhanced mammography generator circuitry 401 performs contrast enhanced mammography as described in connection with FIG. 7. In some examples, the operations of block 642 are skipped. After block 642, control advances to block 644.

At block 644, the network interface 252 transmits the completed medical examination to a radiology system. For example, if an X-ray examination and an ultrasound examination are completed for a specific patient, then the network interface 252 transmits the completed X-ray examination and the completed ultrasound examination to the radiology system (e.g., a radiology viewer, other workstation, etc.). By transmitting completed examinations, the radiology system is able to diagnose the patient without requesting additional medical images. After block 644, the instructions 600 end.

FIG. 7 is a flowchart representative of example machine readable instructions and/or example operations 642 that may be executed, instantiated, and/or performed by programmable circuitry to perform contrast enhanced mammography. The example machine-readable instructions and/or the example operations 642 of FIG. 7 begin at block 702. At block 702, the image management circuitry 316 determines if a prior MRI exists. For example, if the image management circuitry 316 determines that a prior MRI exists (e.g., “YES”), control advances to block 704. Alternatively, if the image management circuitry 316 does not determine that a prior MRI exists (e.g., “NO”), control advances to block 706. The example image management circuitry 316 may determine that a prior MRI exists by querying the local patient data store 270.

At block 704, the image management circuitry 316 loads the prior MRI into the analyzer circuitry 318. The example image management circuitry 316 accesses prior MRI from the example local patient data store 270. Control advances to block 716.

At block 706, the image management circuitry 316 determines to either capture a two-dimensional image or a three-dimensional image. For example, if the image management circuitry 316 determines to capture a two-dimensional image (e.g., “2D”), control advances to block 708. Alternatively, if the image management circuitry 316 determines to capture a three-dimensional image (e.g., 3D″), control advances to block 712.

At block 708, the CC CEM generator circuitry 402 and/or the MLO CEM generator circuitry 404 performs the CC contrast enhanced mammography and/or MLO contrast enhanced mammography to generate two-dimensional images. By performing the CC contrast enhanced mammography, the CC CEM generator circuitry 402 captures a cranial-caudal image by aligning the source 102 (FIG. 1A) over the breast in a cranial-caudal orientation. By performing the MLO contrast enhanced mammography, the MLO CEM generator circuitry 404 rotates the source 102 (FIG. 1A) to be angled over the breast to generate a mediolateral oblique view. Control advances to block 710.

At block 710, the image management circuitry 316 loads the two-dimensional images (e.g., the CC mammography images and/or the MLO contrast enhanced mammography images) into the analyzer circuitry 318. The image management circuitry 316 loads the two-dimensional images from the local patient data store 270. Control advances to block 716.

At block 712, the CEDBT generator circuitry 406 performs contrast-enhanced digital breast tomosynthesis (CEDBT) to generate three-dimensional images. By performing contrast-enhanced DBT, the CEDBT generator circuitry 406 generates three-dimensional DBT images before contrast is applied in the tissue of the patient and after contrast is applied in the tissue of the patient. Control advances to block 714.

At block 714, the image management circuitry 316 loads the three-dimensional images into the analyzer circuitry 318. The image management circuitry 316 loads the three-dimensional images from the local patient data store 270. Control advances to block 716.

At block 716, the analyzer circuitry 318 analyzes the images. In some examples, the images are at least one of the following types: two-dimensional images, three-dimensional images, or prior MRI images. The analyzer circuitry 318 determines if there are any findings in the images. For example, a finding may be suspicious or non-suspicious. In some examples, the AI model inference circuitry 256 analyzes the images. By analyzing the images, the analyzer circuitry 318 or the AI model inference circuitry 256 determines if a late CEM image is useful to take. For example, certain cancers are known to quickly absorb the contrast. In such examples, if one of these cancers is predicted to be in the breast of the patient, then a late CEM image is unlikely to be useful. However, in other examples, other cancers are known to become more easily identified after a late CEM image. In such examples, a late CEM image is likely to be useful. While a late CEM image may be unlikely to be useful, determining a type of cancer that quickly absorbs the contrast is useful. For example, determining that a patient has a lesion with a rapid washout curve (e.g., trapeze) or determining that a patient has a lesion with increasing contrast (e.g., ramp) is useful in diagnosis and/or development of a treatment plan. In some examples, the analyzer circuitry 318 or the AI model inference circuitry 256 determines if a late CEM image is likely to be useful based on a confidence to a pattern. For example, if the analyzer circuitry 318 or the AI model inference circuitry 256 determines that a set of tissues is cancer, there is no need to perform the late CEM. Alternatively, if the analyzer circuitry 318 or the AI model inference circuitry 256 is unable to determine if the set of tissues is cancer, then there is a need to perform the late CEM.

At block 718, the BPE analyzer circuitry 314 analyzes the kinetic of the BPE in the images. For example, the BPE analyzer circuitry 314 determines an absorption rate of the background parenchymal serum to determine if the background parenchymal serum enhances the images. By determining an absorption rate, the BPE analyzer circuitry 314 determines other facts of the breasts of the patients. For example, the BPE analyzer circuitry 314 accesses an AI/ML model that analyzes one or multiple CEM images of the same breast to provide a BPE class. The BPE classes include minimal, mild, moderate, and marked (e.g., BI-RADS classification). The CEM AI/ML model is trained on CEM images which were designated a BPE class (e.g., labeled training data) as ground truth. By analyzing the kinetic of the BPE, the BPE analyzer circuitry 314 compares the absorption of the iodine with dual energy analysis. In some examples, the BPE analyzer circuitry 314 determines if a first breast absorbs contrast symmetrically with a second breast. If there is a deviation from the symmetric absorption of contrast, the BPE analyzer circuitry 314 determines that there may be cancer in one of the breasts. In some examples, the BPE analyzer circuitry 314 uses the symmetrical analysis in the presence of lesions that are faint. Control advances to block 720.

At block 720, the findings circuitry 306 analyzes the presence of a suspicious finding. For example, the findings circuitry 306 determines that a suspicious finding exists in the tissue if there is a white mass that exceeds a certain diameter. Control advances to block 722.

At block 722, the analyzer circuitry 318 analyzes the kinetic of a suspicious finding. For example, the findings circuitry 306 determines if there is absorption or movement in a suspicious finding. Control advances to block 724.

At block 724, the analyzer circuitry 318 generates a recommendation. For example, the recommendation includes an indication if a contrast enhanced mammography (CEM) two-dimensional (2D) image or contrast enhanced mammography (CEM) three-dimensional (3D) image is determined to be useful. For example, the analyzer circuitry 318, based on the image analysis, the BPE kinetic analysis, presence or absence of suspicious findings, and kinetics for suspicious findings, generates a recommendation. In some examples, the analyzer circuitry 318 uses at least one of image analysis, the BPE kinetic analysis, presence or absence of suspicious findings, and kinetics for suspicious findings to generate the recommendation. In some examples, the recommendation includes different examinations such as the examinations of FIG. 6, in addition to the indication if a contrast enhanced mammography (CEM) image is useful or superfluous. Control advances to block 726.

At block 726, the analyzer circuitry 318 determines if a contrast enhanced mammography image would be useful. For example, if the analyzer circuitry 318 determines that contrast enhanced mammography is useful (e.g., “YES”), control advances to block 728. Alternatively, if the analyzer circuitry 318 determines that contrast enhanced mammography is not useful (e.g., “NO”), control advances to block 732.

At block 728, the analyzer circuitry 318 outputs a positive response. For example, the positive response corresponds to an indication that a subsequent image (e.g., a late contrast enhanced mammography image) is useful. This subsequent contrast enhanced mammography image may be a two-dimensional image or a three-dimensional DBT image. Control advances to block 730.

At block 730, the timer circuitry 266 generates a timing of when to perform the CEM image. For example, a timing may be a short amount of time on the same day (e.g., immediately, 5 minutes, 10 minutes, etc.), a medium amount of time (e.g., a subsequent day or week), or a long amount of time (e.g., a later month or year). In some examples, the timer circuitry 266 generates a first time to take the capture the CEM image and a second time to analyze the captured CEM image. In such examples, the iodine is absorbed over a first time period (e.g., four minutes from injection), is brightest in the tissue of the patient for a second time period (e.g., six minutes from injection), before fading away in the third time period (e.g., twelve minutes from injection). Therefore, the timer circuitry 266 indicates optimal times for the contrast to be analyzed. Control advances to block 734.

At block 732, the analyzer circuitry 318 outputs a negative response. For example, the negative court response corresponds to a determination that a late contrast enhanced mammography image would not be useful. Control advances to block 734.

At block 734, the image management circuitry 316 determines to take a subsequent image. For example, if the image management circuitry 316 determines to take a subsequent image (e.g., “YES”), control returns to block 702. Alternatively, if the image management circuitry 316 determines to not take a subsequent image (e.g., “NO”), the instructions 642 end.

FIG. 8 is a flowchart representative of example machine readable instructions and/or example operations 800 that may be executed, instantiated, and/or performed by programmable circuitry to generate the image capture protocol and transmit the image capture protocol to a medical imaging device. FIG. 8 follows the breast imaging processing circuitry 110 of FIG. 2. The breast imaging processing circuitry 110 is in communication with the medical device circuitry 250 of the example medical imaging device 100. In some examples, the operations of FIG. 8 are performed by the breast imaging protocol circuitry 126 (FIG. 1D).

The example machine-readable instructions and/or the example operations 800 of FIG. 8 begin at block 802, the image capture circuitry 208 of the breast imaging processing circuitry 110 generates an image capture protocol. For example, the image capture protocol (e.g., image capture instruction set, decision tree, workflow management architecture, image capture plan, etc.) instructs the medical imaging devices 100A, 100B, 100C, 100D, 100E of FIG. 1C on which medical images (e.g., ultrasound, X-ray, two-dimensional, three-dimensional, contrast-enhanced, standard etc.) to capture. In addition, the image capture protocol includes different times (e.g., different scenarios) that correspond to different medical images that are to be captured. By capturing all the medical images without intervention from a radiologist (e.g., analysis from a radiologist), the medical device of FIG. 1 saves processor cycles by transmitting one package of medical images and/or medical examinations, rather than sending a first medical image, receiving an instruction from the radiologist to capture a second medical image, and then capturing the second medical image, and then transmitting the second medical image to the radiologist.

At block 804, the network interface 202 of the breast imaging processing circuitry 110 sends the image capture protocol to hospitals. By sending the image capture protocol to hospitals, the network interface 202 sends the image capture protocol to medical device circuitry 250 that is implemented on the example medical imaging device 100 of FIG. 1A.

At block 806, the network interface 202 accesses hospital specific information. For example, a radiologist confirms data (e.g., a number of medical imaging devices in the hospital, available medical device types (e.g., ultrasound is available, but X-ray is not), a number of available doctors, and a number of examination rooms) to generate the hospital specific information. By accessing the hospital specific information, the network interface 202 allows the updater circuitry 210 to update the image capture protocol to apply to the specific equipment and personnel available at the hospital.

At block 808, the updater circuitry 210 determines to update the image capture protocol. For example, if the updater circuitry 210 determines to update the image capture protocol (e.g., “YES”), control advances to block 810. Alternatively, if the updater circuitry 210 determines to not update the image capture protocol (e.g., “NO”), control advances to block 812. The example updater circuitry 210 determines to update the image capture protocol based on a threshold of time that has elapsed since the network interface 202 accessed the hospital-information. For example, if the network interface 202 accesses the hospital-information earlier that week, the updater circuitry 210 does not determine to update the image capture protocol. Alternatively, if the network interface 202 accesses the hospital information from a year ago, the updater circuitry 210 determines to update the image capture protocol with the recent hospital information.

At block 810, the updater circuitry 210 updates the image capture protocol based on hospital specific information. For example, if a hospital only has X-ray machines as the medical imaging devices, then protocol block (e.g., path, decision, leaves) that leads to taking an ultrasound will be removed from the image capture protocol. Control advances to block 812.

At block 812, the updater circuitry 210 marks the image capture protocol as ready for loading into the medical imaging device 100. By marking the image capture protocol as ready for loading, the medical device circuitry 250 of the medical imaging device 100 is able to load the image capture protocol with the protocol executor circuitry 258. After block 812, the instructions 800 end.

FIG. 9 is a flowchart representative of example machine readable instructions and/or example operations 900 that may be executed, instantiated, and/or performed by programmable circuitry to capture mammographic images by following the image capture protocol. The example machine-readable instructions and/or the example operations 900 of FIG. 9 begin at block 902, at which the protocol executor circuitry 258 loads the image capture protocol. In the example of FIG. 9, the image capture protocol is the image capture protocol from FIG. 8 which has been marked for loading which includes hospital-specific information. In other examples, the protocol executor circuitry 258 uploads an image capture protocol that has not been marked for uploading.

At block 904, the medical device circuitry 250 executes the operations of the image capture protocol. For example, the network interface 252, the AI model training circuitry 254, the AI model inference circuitry 256, the protocol executor circuitry 258, the updater circuitry 260, the image processor circuitry 262, the image generator circuitry 264, the timer circuitry 266, the user interface circuitry 274, and the trigger circuitry 276 are all used in executing the operations of the image capture protocol (e.g., such as the flowcharts of FIGS. 6A-6B and FIG. 7). The image capture protocol may also use the subcomponents that include the image positioning circuitry 302, image quality circuitry 304, findings circuitry 306, density assessment circuitry 308, risk score calculator circuitry 310, Breast-Imaging Reporting and Data System (BIRAD) score calculator circuitry 312, Background Parenchymal Enhancement (BPE) analyzer circuitry 314, image management circuitry 316, and analyzer circuitry 318 of the image processor circuitry 262. The image capture protocol may also use the subcomponents cranial-caudal (CC) contrast enhanced mammography (CEM) generator 402, a mediolateral oblique (MLO) CEM generator 404, contrasted-enhanced (CE) digital breast tomosynthesis (DBT) generation circuitry 406, a mediolateral (ML) CEM generator 408, ultrasound generator circuitry 410, magnified image circuitry 412, a spot examination circuitry 414, and magnetic resonance imaging (MRI) generator circuitry 416, and DBT generator circuitry 418 of the image generator circuitry 264.

At block 906, the user interface circuitry 274 determines to alert an external system (e.g., presents a notification on a display that may be viewed by a user of the medical device). For example, if the user interface circuitry 274 determines to alert the external system (e.g., “YES”), control advances to block 908. Alternatively, if the user interface circuitry 274 determines to not alert the external system (e.g., “NO”), control advances to block 910. The user interface circuitry 274 may determine to notify a user that an image is to be retaken or of the next operation to perform. This image that is to be retaken may require movements of the patient so that the breast is fully in view of the detector of the medical imaging device 100A, 100B, 100C, 100D, 100E.

At block 908, the user interface circuitry 274 notifies a user to reposition the patient. For example, by notifying a user (e.g., technologist) to reposition the patient, the medical device circuitry 250 does not transmit blurry images to the external device of the radiologist which saves processor cycles. Control advances to block 910.

At block 910, the medical device circuitry 250 continues the operations of the image capture protocol. For example, these operations can include further types of images to be captured or further repositioning of the patient.

At block 912, the protocol executor circuitry 258 determines if exams were generated. For example, if the protocol executor circuitry 258 determines that exams were generated (e.g., “YES”), control advances to block 916. Alternatively, if the protocol executor circuitry 258 determines that exams were not generated (e.g., “NO”), control advances to block 914.

At block 914, the user interface circuitry notifies a user that no exams were generated. For example, the analyzer circuitry 318 during the operations of the image capture protocol may determine that no ultrasound or MRI is needed. After block 914 control advances to block 924.

At block 916, the protocol executor circuitry 258 determines how to send the exams that were generated.

At block 918, the follower circuitry determines to send the exams automatically. For example, if the protocol executor circuitry 258 determines to send the exams automatically (e.g., “YES”), control advances to block 920. Alternatively, if the protocol executor circuitry 258 determines to not send the exams automatically (e.g., “NO”), control advances to block 922. After the steps of the image capture protocol are completed, all the images of all the examinations are sent to the radiology system for review and/or processing.

At block 920, the network interface 252 sends the exams automatically to a radiology system. Control advances to block 924.

At block 922, the user interface circuitry 274 is operated by a user to send the exams manually. Control advances to block 924.

At block 924, the protocol executor circuitry 258 determines to return. For example, if the protocol executor circuitry 258 determines to return (e.g., “YES”), control returns to block 902. Alternatively, if the protocol executor circuitry 258 determines to not return (e.g., “NO”), the instructions 900 end.

FIG. 10 is a block diagram of an example programmable circuitry platform 1000 structured to execute and/or instantiate the example machine-readable instructions and/or the example operations of FIGS. 5-9 to implement the medical device circuitry 250 of FIG. 2 and/or the breast imaging processing circuitry 110 of FIG. 2. The programmable circuitry platform 1000 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, or any other type of computing and/or electronic device.

The programmable circuitry platform 1000 of the illustrated example includes programmable circuitry 1012. The programmable circuitry 1012 of the illustrated example is hardware. For example, the programmable circuitry 1012 can be implemented by one or more integrated circuits, logic circuits, FPGAs, microprocessors, CPUs, GPUs, DSPs, and/or microcontrollers from any desired family or manufacturer. The programmable circuitry 1012 may be implemented by one or more semiconductor based (e.g., silicon based) devices. In this example, the programmable circuitry 1012 implements the network interface 202, artificial intelligence (AI) model training circuitry 204, AI model inference circuitry 206, image capture circuitry 208, updater circuitry 210, image processor circuitry 212, network interface 252, AI model training circuitry 254, AI model inference circuitry 256, protocol executor circuitry 258, updater circuitry 260, image processor circuitry 262, image generator circuitry 264, timer circuitry 266, image positioning circuitry 302, image quality circuitry 304, findings circuitry 306, density assessment circuitry 308, risk score calculator circuitry 310, Breast-Imaging Reporting and Data System (BIRAD) score calculator circuitry 312, Background Parenchymal Enhancement (BPE) analyzer circuitry 314, image management circuitry 316, analyzer circuitry 318, the cranial-caudal (CC) contrast enhanced mammography (CEM) generator 402, a mediolateral oblique (MLO) CEM generator 404, contrasted-enhanced (CE) digital breast tomosynthesis (DBT) generation circuitry 406, a mediolateral (ML) CEM generator 408, ultrasound generator circuitry 410, magnified image circuitry 412, a spot examination circuitry 414, and magnetic resonance imaging (MRI) generator circuitry 416, and DBT generator circuitry 418.

The programmable circuitry 1012 of the illustrated example includes a local memory 1013 (e.g., a cache, registers, etc.). The programmable circuitry 1012 of the illustrated example is in communication with main memory 1014, 1016, which includes a volatile memory 1014 and a non-volatile memory 1016, by a bus 1018. The volatile memory 1014 may be implemented by Synchronous Dynamic Random Access Memory (SDRAM), Dynamic Random Access Memory (DRAM), RAMBUS® Dynamic Random Access Memory (RDRAM®), and/or any other type of RAM device. The non-volatile memory 1016 may be implemented by flash memory and/or any other desired type of memory device. Access to the main memory 1014, 1016 of the illustrated example is controlled by a memory controller 1017. In some examples, the memory controller 1017 may be implemented by one or more integrated circuits, logic circuits, microcontrollers from any desired family or manufacturer, or any other type of circuitry to manage the flow of data going to and from the main memory 1014, 1016.

The programmable circuitry platform 1000 of the illustrated example also includes interface circuitry 1020. The interface circuitry 1020 may be implemented by hardware in accordance with any type of interface standard, such as an Ethernet interface, a universal serial bus (USB) interface, a Bluetooth® interface, a near field communication (NFC) interface, a Peripheral Component Interconnect (PCI) interface, and/or a Peripheral Component Interconnect Express (PCIe) interface.

In the illustrated example, one or more input devices 1022 are connected to the interface circuitry 1020. The input device(s) 1022 permit(s) a user (e.g., a human user, a machine user, etc.) to enter data and/or commands into the programmable circuitry 1012. The input device(s) 1022 can be implemented by, for example, an audio sensor, a microphone, a camera (still or video), a keyboard, a button, a mouse, a touchscreen, a trackpad, a trackball, an isopoint device, and/or a voice recognition system.

One or more output devices 1024 are also connected to the interface circuitry 1020 of the illustrated example. The output device(s) 1024 can be implemented, for example, by display devices (e.g., a light emitting diode (LED), an organic light emitting diode (OLED), a liquid crystal display (LCD), a cathode ray tube (CRT) display, an in-place switching (IPS) display, a touchscreen, etc.), a tactile output device, a printer, and/or speaker. The interface circuitry 1020 of the illustrated example, thus, typically includes a graphics driver card, a graphics driver chip, and/or graphics processor circuitry such as a GPU.

The interface circuitry 1020 of the illustrated example also includes a communication device such as a transmitter, a receiver, a transceiver, a modem, a residential gateway, a wireless access point, and/or a network interface to facilitate exchange of data with external machines (e.g., computing devices of any kind) by a network 1026. The communication can be by, for example, an Ethernet connection, a digital subscriber line (DSL) connection, a telephone line connection, a coaxial cable system, a satellite system, a beyond-line-of-sight wireless system, a line-of-sight wireless system, a cellular telephone system, an optical connection, etc.

The programmable circuitry platform 1000 of the illustrated example also includes one or more mass storage discs or devices 1028 to store firmware, software, and/or data. Examples of such mass storage discs or devices 1028 include magnetic storage devices (e.g., floppy disk, drives, HDDs, etc.), optical storage devices (e.g., Blu-ray disks, CDs, DVDs, etc.), RAID systems, and/or solid-state storage discs or devices such as flash memory devices and/or SSDs.

The machine readable instructions 1032, which may be implemented by the machine readable instructions of FIGS. 5-9, may be stored in the mass storage device 1028, in the volatile memory 1014, in the non-volatile memory 1016, and/or on at least one non-transitory computer readable storage medium such as a CD or DVD which may be removable.

FIG. 11 is a block diagram of an example implementation of the programmable circuitry 1012 of FIG. 10. In this example, the programmable circuitry 1012 of FIG. 10 is implemented by a microprocessor 1100. For example, the microprocessor 1100 may be a general-purpose microprocessor (e.g., general-purpose microprocessor circuitry). The microprocessor 1100 executes some or all of the machine-readable instructions of the flowcharts of FIGS. 5-9 to effectively instantiate the circuitry of FIG. 2 as logic circuits to perform operations corresponding to those machine readable instructions. In some such examples, the circuitry of FIG. 2 is instantiated by the hardware circuits of the microprocessor 1100 in combination with the machine-readable instructions. For example, the microprocessor 1100 may be implemented by multi-core hardware circuitry such as a CPU, a DSP, a GPU, an XPU, etc. Although it may include any number of example cores 1102 (e.g., 1 core), the microprocessor 1100 of this example is a multi-core semiconductor device including N cores. The cores 1102 of the microprocessor 1100 may operate independently or may cooperate to execute machine readable instructions. For example, machine code corresponding to a firmware program, an embedded software program, or a software program may be executed by one of the cores 1102 or may be executed by multiple ones of the cores 1102 at the same or different times. In some examples, the machine code corresponding to the firmware program, the embedded software program, or the software program is split into threads and executed in parallel by two or more of the cores 1102. The software program may correspond to a portion or all of the machine readable instructions and/or operations represented by the flowcharts of FIGS. 5-9.

The cores 1102 may communicate by a first example bus 1104. In some examples, the first bus 1104 may be implemented by a communication bus to effectuate communication associated with one(s) of the cores 1102. For example, the first bus 1104 may be implemented by at least one of an Inter-Integrated Circuit (I2C) bus, a Serial Peripheral Interface (SPI) bus, a PCI bus, or a PCIe bus. Additionally or alternatively, the first bus 1104 may be implemented by any other type of computing or electrical bus. The cores 1102 may obtain data, instructions, and/or signals from one or more external devices by example interface circuitry 1106. The cores 1102 may output data, instructions, and/or signals to the one or more external devices by the interface circuitry 1106. Although the cores 1102 of this example include example local memory 1120 (e.g., Level 1 (L1) cache that may be split into an L1 data cache and an L1 instruction cache), the microprocessor 1100 also includes example shared memory 1110 that may be shared by the cores (e.g., Level 2 (L2 cache)) for high-speed access to data and/or instructions. Data and/or instructions may be transferred (e.g., shared) by writing to and/or reading from the shared memory 1110. The local memory 1120 of each of the cores 1102 and the shared memory 1110 may be part of a hierarchy of storage devices including multiple levels of cache memory and the main memory (e.g., the main memory 1014, 1016 of FIG. 10). Typically, higher levels of memory in the hierarchy exhibit lower access time and have smaller storage capacity than lower levels of memory. Changes in the various levels of the cache hierarchy are managed (e.g., coordinated) by a cache coherency policy.

Each core 1102 may be referred to as a CPU, DSP, GPU, etc., or any other type of hardware circuitry. Each core 1102 includes control unit circuitry 1114, arithmetic and logic (AL) circuitry (sometimes referred to as an ALU) 1116, a plurality of registers 1118, the local memory 1120, and a second example bus 1122. Other structures may be present. For example, each core 1102 may include vector unit circuitry, single instruction multiple data (SIMD) unit circuitry, load/store unit (LSU) circuitry, branch/jump unit circuitry, floating-point unit (FPU) circuitry, etc. The control unit circuitry 1114 includes semiconductor-based circuits structured to control (e.g., coordinate) data movement within the corresponding core 1102. The AL circuitry 1116 includes semiconductor-based circuits structured to perform one or more mathematic and/or logic operations on the data within the corresponding core 1102. The AL circuitry 1116 of some examples performs integer based operations. In other examples, the AL circuitry 1116 also performs floating-point operations. In yet other examples, the AL circuitry 1116 may include first AL circuitry that performs integer-based operations and second AL circuitry that performs floating-point operations. In some examples, the AL circuitry 1116 may be referred to as an Arithmetic Logic Unit (ALU).

The registers 1118 are semiconductor-based structures to store data and/or instructions such as results of one or more of the operations performed by the AL circuitry 1116 of the corresponding core 1102. For example, the registers 1118 may include vector register(s), SIMD register(s), general-purpose register(s), flag register(s), segment register(s), machine-specific register(s), instruction pointer register(s), control register(s), debug register(s), memory management register(s), machine check register(s), etc. The registers 1118 may be arranged in a bank as shown in FIG. 11. Alternatively, the registers 1118 may be organized in any other arrangement, format, or structure, such as by being distributed throughout the core 1102 to shorten access time. The second bus 1122 may be implemented by at least one of an I2C bus, a SPI bus, a PCI bus, or a PCIe bus.

Each core 1102 and/or, more generally, the microprocessor 1100 may include additional and/or alternate structures to those shown and described above. For example, one or more clock circuits, one or more power supplies, one or more power gates, one or more cache home agents (CHAs), one or more converged/common mesh stops (CMSs), one or more shifters (e.g., barrel shifter(s)) and/or other circuitry may be present. The microprocessor 1100 is a semiconductor device fabricated to include many transistors interconnected to implement the structures described above in one or more integrated circuits (ICs) contained in one or more packages.

The microprocessor 1100 may include and/or cooperate with one or more accelerators (e.g., acceleration circuitry, hardware accelerators, etc.). In some examples, accelerators are implemented by logic circuitry to perform certain tasks more quickly and/or efficiently than can be done by a general-purpose processor. Examples of accelerators include ASICs and FPGAs such as those discussed herein. A GPU, DSP and/or other programmable device can also be an accelerator. Accelerators may be on-board the microprocessor 1100, in the same chip package as the microprocessor 1100 and/or in one or more separate packages from the microprocessor 1100.

FIG. 12 is a block diagram of another example implementation of the programmable circuitry 1012 of FIG. 10. In this example, the programmable circuitry 1012 is implemented by FPGA circuitry 1200. For example, the FPGA circuitry 1200 may be implemented by an FPGA. The FPGA circuitry 1200 can be used, for example, to perform operations that could otherwise be performed by the example microprocessor 1100 of FIG. 11 executing corresponding machine readable instructions. However, once configured, the FPGA circuitry 1200 instantiates the operations and/or functions corresponding to the machine readable instructions in hardware and, thus, can often execute the operations/functions faster than they could be performed by a general-purpose microprocessor executing the corresponding software.

More specifically, in contrast to the microprocessor 1100 of FIG. 11 described above (which is a general purpose device that may be programmed to execute some or all of the machine readable instructions represented by the flowchart(s) of FIGS. 5-9 but whose interconnections and logic circuitry are fixed once fabricated), the FPGA circuitry 1200 of the example of FIG. 12 includes interconnections and logic circuitry that may be configured, structured, programmed, and/or interconnected in different ways after fabrication to instantiate, for example, some or all of the operations/functions corresponding to the machine readable instructions represented by the flowchart(s) of FIGS. 5-9. In particular, the FPGA circuitry 1200 may be thought of as an array of logic gates, interconnections, and switches. The switches can be programmed to change how the logic gates are interconnected by the interconnections, effectively forming one or more dedicated logic circuits (unless and until the FPGA circuitry 1200 is reprogrammed). The configured logic circuits enable the logic gates to cooperate in different ways to perform different operations on data received by input circuitry. Those operations may correspond to some or all of the instructions (e.g., the software and/or firmware) represented by the flowchart(s) of FIGS. 5-9. As such, the FPGA circuitry 1200 may be configured and/or structured to effectively instantiate some or all of the operations/functions corresponding to the machine readable instructions of the flowchart(s) of FIGS. 5-9 as dedicated logic circuits to perform the operations/functions corresponding to those software instructions in a dedicated manner analogous to an ASIC. Therefore, the FPGA circuitry 1200 may perform the operations/functions corresponding to the some or all of the machine readable instructions of FIGS. 5-9 faster than the general-purpose microprocessor can execute the same.

In the example of FIG. 12, the FPGA circuitry 1200 is configured and/or structured in response to being programmed (and/or reprogrammed one or more times) based on a binary file. In some examples, the binary file may be compiled and/or generated based on instructions in a hardware description language (HDL) such as Lucid, Very High Speed Integrated Circuits (VHSIC) Hardware Description Language (VHDL), or Verilog. For example, a user (e.g., a human user, a machine user, etc.) may write code or a program corresponding to one or more operations/functions in an HDL; the code/program may be translated into a low-level language as needed; and the code/program (e.g., the code/program in the low-level language) may be converted (e.g., by a compiler, a software application, etc.) into the binary file. In some examples, the FPGA circuitry 1200 of FIG. 12 may access and/or load the binary file to cause the FPGA circuitry 1200 of FIG. 12 to be configured and/or structured to perform the one or more operations/functions. For example, the binary file may be implemented by a bit stream (e.g., one or more computer-readable bits, one or more machine-readable bits, etc.), data (e.g., computer-readable data, machine-readable data, etc.), and/or machine-readable instructions accessible to the FPGA circuitry 1200 of FIG. 12 to cause configuration and/or structuring of the FPGA circuitry 1200 of FIG. 12, or portion(s) thereof.

In some examples, the binary file is compiled, generated, transformed, and/or otherwise output from a uniform software platform utilized to program FPGAs. For example, the uniform software platform may translate first instructions (e.g., code or a program) that correspond to one or more operations/functions in a high-level language (e.g., C, C++, Python, etc.) into second instructions that correspond to the one or more operations/functions in an HDL. In some such examples, the binary file is compiled, generated, and/or otherwise output from the uniform software platform based on the second instructions. In some examples, the FPGA circuitry 1200 of FIG. 12 may access and/or load the binary file to cause the FPGA circuitry 1200 of FIG. 12 to be configured and/or structured to perform the one or more operations/functions. For example, the binary file may be implemented by a bit stream (e.g., one or more computer-readable bits, one or more machine-readable bits, etc.), data (e.g., computer-readable data, machine-readable data, etc.), and/or machine-readable instructions accessible to the FPGA circuitry 1200 of FIG. 12 to cause configuration and/or structuring of the FPGA circuitry 1200 of FIG. 12, or portion(s) thereof.

The FPGA circuitry 1200 of FIG. 12, includes example input/output (I/O) circuitry 1202 to obtain and/or output data to/from example configuration circuitry 1204 and/or external hardware 1206. For example, the configuration circuitry 1204 may be implemented by interface circuitry that may obtain a binary file, which may be implemented by a bit stream, data, and/or machine-readable instructions, to configure the FPGA circuitry 1200, or portion(s) thereof. In some such examples, the configuration circuitry 1204 may obtain the binary file from a user, a machine (e.g., hardware circuitry (e.g., programmable or dedicated circuitry) that may implement an Artificial Intelligence/Machine Learning (AI/ML) model to generate the binary file), etc., and/or any combination(s) thereof). In some examples, the external hardware 1206 may be implemented by external hardware circuitry. For example, the external hardware 1206 may be implemented by the microprocessor 1100 of FIG. 11.

The FPGA circuitry 1200 also includes an array of example logic gate circuitry 1208, a plurality of example configurable interconnections 1210, and example storage circuitry 1212. The logic gate circuitry 1208 and the configurable interconnections 1210 are configurable to instantiate one or more operations/functions that may correspond to at least some of the machine readable instructions of FIGS. 5-9 and/or other desired operations. The logic gate circuitry 1208 shown in FIG. 12 is fabricated in blocks or groups. Each block includes semiconductor-based electrical structures that may be configured into logic circuits. In some examples, the electrical structures include logic gates (e.g., And gates, Or gates, Nor gates, etc.) that provide basic building blocks for logic circuits. Electrically controllable switches (e.g., transistors) are present within each of the logic gate circuitry 1208 to enable configuration of the electrical structures and/or the logic gates to form circuits to perform desired operations/functions. The logic gate circuitry 1208 may include other electrical structures such as look-up tables (LUTs), registers (e.g., flip-flops or latches), multiplexers, etc.

The configurable interconnections 1210 of the illustrated example are conductive pathways, traces, vias, or the like that may include electrically controllable switches (e.g., transistors) whose state can be changed by programming (e.g., using an HDL instruction language) to activate or deactivate one or more connections between one or more of the logic gate circuitry 1208 to program desired logic circuits.

The storage circuitry 1212 of the illustrated example is structured to store result(s) of the one or more of the operations performed by corresponding logic gates. The storage circuitry 1212 may be implemented by registers or the like. In the illustrated example, the storage circuitry 1212 is distributed amongst the logic gate circuitry 1208 to facilitate access and increase execution speed.

The example FPGA circuitry 1200 of FIG. 12 also includes example dedicated operations circuitry 1214. In this example, the dedicated operations circuitry 1214 includes special purpose circuitry 1216 that may be invoked to implement commonly used functions to avoid the need to program those functions in the field. Examples of such special purpose circuitry 1216 include memory (e.g., DRAM) controller circuitry, PCIe controller circuitry, clock circuitry, transceiver circuitry, memory, and multiplier-accumulator circuitry. Other types of special purpose circuitry may be present. In some examples, the FPGA circuitry 1200 may also include example general purpose programmable circuitry 1218 such as an example CPU 1220 and/or an example DSP 1222. Other general purpose programmable circuitry 1218 may additionally or alternatively be present such as a GPU, an XPU, etc., that can be programmed to perform other operations.

Although FIGS. 11 and 12 illustrate two example implementations of the programmable circuitry 1012 of FIG. 10, many other approaches are contemplated. For example, FPGA circuitry may include an on-board CPU, such as one or more of the example CPU 1220 of FIG. 11. Therefore, the programmable circuitry 1012 of FIG. 10 may additionally be implemented by combining at least the example microprocessor 1100 of FIG. 11 and the example FPGA circuitry 1200 of FIG. 12. In some such hybrid examples, one or more cores 1102 of FIG. 11 may execute a first portion of the machine readable instructions represented by the flowchart(s) of FIGS. 5-9 to perform first operation(s)/function(s), the FPGA circuitry 1200 of FIG. 12 may be configured and/or structured to perform second operation(s)/function(s) corresponding to a second portion of the machine readable instructions represented by the flowcharts of FIG. 5-9, and/or an ASIC may be configured and/or structured to perform third operation(s)/function(s) corresponding to a third portion of the machine readable instructions represented by the flowcharts of FIGS. 5-9.

It should be understood that some or all of the circuitry of FIG. 2 may, thus, be instantiated at the same or different times. For example, same and/or different portion(s) of the microprocessor 1100 of FIG. 11 may be programmed to execute portion(s) of machine-readable instructions at the same and/or different times. In some examples, same and/or different portion(s) of the FPGA circuitry 1200 of FIG. 12 may be configured and/or structured to perform operations/functions corresponding to portion(s) of machine-readable instructions at the same and/or different times.

In some examples, some or all of the circuitry of FIG. 2 may be instantiated, for example, in one or more threads executing concurrently and/or in series. For example, the microprocessor 1100 of FIG. 11 may execute machine readable instructions in one or more threads executing concurrently and/or in series. In some examples, the FPGA circuitry 1200 of FIG. 12 may be configured and/or structured to carry out operations/functions concurrently and/or in series. Moreover, in some examples, some or all of the circuitry of FIG. 2 may be implemented within one or more virtual machines and/or containers executing on the microprocessor 1100 of FIG. 11.

In some examples, the programmable circuitry 1012 of FIG. 10 may be in one or more packages. For example, the microprocessor 1100 of FIG. 11 and/or the FPGA circuitry 1200 of FIG. 12 may be in one or more packages. In some examples, an XPU may be implemented by the programmable circuitry 1012 of FIG. 10, which may be in one or more packages. For example, the XPU may include a CPU (e.g., the microprocessor 1100 of FIG. 11, the CPU 1220 of FIG. 12, etc.) in one package, a DSP (e.g., the DSP 1222 of FIG. 12) in another package, a GPU in yet another package, and an FPGA (e.g., the FPGA circuitry 1200 of FIG. 12) in still yet another package.

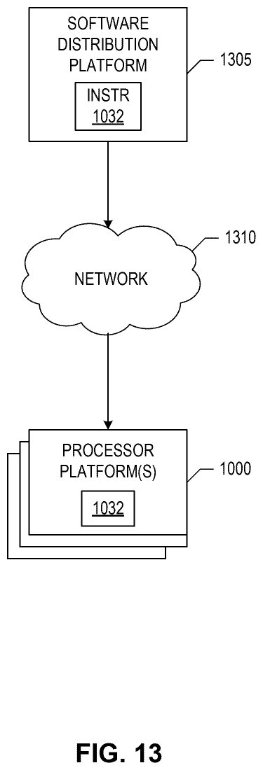

A block diagram illustrating an example software distribution platform 1305 to distribute software such as the example machine readable instructions 1032 of FIG. 10 to other hardware devices (e.g., hardware devices owned and/or operated by third parties from the owner and/or operator of the software distribution platform) is illustrated in FIG. 13. The example software distribution platform 1305 may be implemented by any computer server, data facility, cloud service, etc., capable of storing and transmitting software to other computing devices. The third parties may be customers of the entity owning and/or operating the software distribution platform 1305. For example, the entity that owns and/or operates the software distribution platform 1305 may be a developer, a seller, and/or a licensor of software such as the example machine readable instructions 1032 of FIG. 10. The third parties may be consumers, users, retailers, OEMs, etc., who purchase and/or license the software for use and/or re-sale and/or sub-licensing. In the illustrated example, the software distribution platform 1305 includes one or more servers and one or more storage devices. The storage devices store the machine readable instructions 1032, which may correspond to the example machine readable instructions of FIGS. 5-9, as described above. The one or more servers of the example software distribution platform 1305 are in communication with an example network 1310, which may correspond to any one or more of the Internet and/or any of the example networks described above. In some examples, the one or more servers are responsive to requests to transmit the software to a requesting party as part of a commercial transaction. Payment for the delivery, sale, and/or license of the software may be handled by the one or more servers of the software distribution platform and/or by a third party payment entity. The servers enable purchasers and/or licensors to download the machine readable instructions 1032 from the software distribution platform 1305. For example, the software, which may correspond to the example machine readable instructions of FIG. 5-9, may be downloaded to the example programmable circuitry platform 1000, which is to execute the machine readable instructions 1032 to implement the medical device circuitry 250. In some examples, one or more servers of the software distribution platform 1305 periodically offer, transmit, and/or force updates to the software (e.g., the example machine readable instructions 1032 of FIG. 10) to ensure improvements, patches, updates, etc., are distributed and applied to the software at the end user devices. Although referred to as software above, the distributed “software” could alternatively be firmware.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc., may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, or (7) A with B and with C. As used herein in the context of describing structures, components, items, objects and/or things, the phrase “at least one of A and B” is intended to refer to implementations including any of (1) at least one A, (2) at least one B, or (3) at least one A and at least one B. Similarly, as used herein in the context of describing structures, components, items, objects and/or things, the phrase “at least one of A or B” is intended to refer to implementations including any of (1) at least one A, (2) at least one B, or (3) at least one A and at least one B. As used herein in the context of describing the performance or execution of processes, instructions, actions, activities, etc., the phrase “at least one of A and B” is intended to refer to implementations including any of (1) at least one A, (2) at least one B, or (3) at least one A and at least one B. Similarly, as used herein in the context of describing the performance or execution of processes, instructions, actions, activities, etc., the phrase “at least one of A or B” is intended to refer to implementations including any of (1) at least one A, (2) at least one B, or (3) at least one A and at least one B.

As used herein, singular references (e.g., “a”, “an”, “first”, “second”, etc.) do not exclude a plurality. The term “a” or “an” object, as used herein, refers to one or more of that object. The terms “a” (or “an”), “one or more”, and “at least one” are used interchangeably herein. Furthermore, although individually listed, a plurality of means, elements, or actions may be implemented by, e.g., the same entity or object. Additionally, although individual features may be included in different examples or claims, these may possibly be combined, and the inclusion in different examples or claims does not imply that a combination of features is not feasible and/or advantageous.

As used herein, unless otherwise stated, the term “above” describes the relationship of two parts relative to Earth. A first part is above a second part, if the second part has at least one part between Earth and the first part. Likewise, as used herein, a first part is “below” a second part when the first part is closer to the Earth than the second part. As noted above, a first part can be above or below a second part with one or more of: other parts therebetween, without other parts therebetween, with the first and second parts touching, or without the first and second parts being in direct contact with one another.

As used in this patent, stating that any part (e.g., a layer, film, area, region, or plate) is in any way on (e.g., positioned on, located on, disposed on, or formed on, etc.) another part, indicates that the referenced part is either in contact with the other part, or that the referenced part is above the other part with one or more intermediate part(s) located therebetween.

As used herein, connection references (e.g., attached, coupled, connected, and joined) may include intermediate members between the elements referenced by the connection reference and/or relative movement between those elements unless otherwise indicated. As such, connection references do not necessarily infer that two elements are directly connected and/or in fixed relation to each other. As used herein, stating that any part is in “contact” with another part is defined to mean that there is no intermediate part between the two parts.

Unless specifically stated otherwise, descriptors such as “first,” “second,” “third,” etc., are used herein without imputing or otherwise indicating any meaning of priority, physical order, arrangement in a list, and/or ordering in any way, but are merely used as labels and/or arbitrary names to distinguish elements for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for identifying those elements distinctly within the context of the discussion (e.g., within a claim) in which the elements might, for example, otherwise share a same name.

As used herein, “approximately” and “about” modify their subjects/values to recognize the potential presence of variations that occur in real world applications. For example, “approximately” and “about” may modify dimensions that may not be exact due to manufacturing tolerances and/or other real world imperfections as will be understood by persons of ordinary skill in the art. For example, “approximately” and “about” may indicate such dimensions may be within a tolerance range of +/−10% unless otherwise specified herein.

As used herein “substantially real time” refers to occurrence in a near instantaneous manner recognizing there may be real world delays for computing time, transmission, etc. Thus, unless otherwise specified, “substantially real time” refers to real time+1 second.

As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

As used herein, “programmable circuitry” is defined to include (i) one or more special purpose electrical circuits (e.g., an application specific circuit (ASIC)) structured to perform specific operation(s) and including one or more semiconductor-based logic devices (e.g., electrical hardware implemented by one or more transistors), and/or (ii) one or more general purpose semiconductor-based electrical circuits programmable with instructions to perform specific functions(s) and/or operation(s) and including one or more semiconductor-based logic devices (e.g., electrical hardware implemented by one or more transistors). Examples of programmable circuitry include programmable microprocessors such as Central Processor Units (CPUs) that may execute first instructions to perform one or more operations and/or functions, Field Programmable Gate Arrays (FPGAs) that may be programmed with second instructions to cause configuration and/or structuring of the FPGAs to instantiate one or more operations and/or functions corresponding to the first instructions, Graphics Processor Units (GPUs) that may execute first instructions to perform one or more operations and/or functions, Digital Signal Processors (DSPs) that may execute first instructions to perform one or more operations and/or functions, XPUs, Network Processing Units (NPUs) one or more microcontrollers that may execute first instructions to perform one or more operations and/or functions and/or integrated circuits such as Application Specific Integrated Circuits (ASICs). For example, an XPU may be implemented by a heterogeneous computing system including multiple types of programmable circuitry (e.g., one or more FPGAs, one or more CPUs, one or more GPUs, one or more NPUs, one or more DSPs, etc., and/or any combination(s) thereof), and orchestration technology (e.g., application programming interface(s) (API(s)) that may assign computing task(s) to whichever one(s) of the multiple types of programmable circuitry is/are suited and available to perform the computing task(s).

As used herein integrated circuit/circuitry is defined as one or more semiconductor packages containing one or more circuit elements such as transistors, capacitors, inductors, resistors, current paths, diodes, etc. For example an integrated circuit may be implemented as one or more of an ASIC, an FPGA, a chip, a microchip, programmable circuitry, a semiconductor substrate coupling multiple circuit elements, a system on chip (SoC), etc.

From the foregoing, it will be appreciated that example systems, apparatus, articles of manufacture, and methods have been disclosed that, in some examples, capture mammographic images automatically by following an image capture protocol that does not require involvement of a radiologist. By enabling the medical imaging device to interact with other devices, dynamically adjust protocols, and reduce interactions with an operator (such as a technician or radiologist), the medical imaging device is able to work independently from input from the radiologist for longer periods of time. The medical imaging devices of the techniques described herein use an image capture protocol to automatically determine whether sufficient images have been obtained to diagnose a patient. One type of mammographic image captured is a late contrast enhanced mammographic image that is taken after a time period has elapsed. Disclosed systems, apparatus, articles of manufacture, and methods improve the efficiency of using a computing device by allowing to transmit, if examinations are performed, at least two mammographic images to a radiologist which saves processor cycles. Rather than transmitting a blurry image, and then receiving an instruction to retake the image, the disclosed systems, apparatus, articles of manufacture, and methods can analyze the first image and then automatically take a second image. By transmitting a second image that is sharper than the first image, the disclosed systems, apparatus, articles of manufacture, and methods save processor cycles that would otherwise be wasted. Disclosed systems, apparatus, articles of manufacture, and methods are accordingly directed to one or more improvement(s) in the operation of a machine such as a computer or other electronic and/or mechanical device. The disclosed systems, apparatus, articles of manufacture, and methods are able to determine the density of patient breasts, and, for certain patient breast density, determine not to perform any examinations, which save healthcare resources and computer resources.
Example 1 includes an apparatus comprising network interface, machine readable instructions, and programmable circuitry to at least one of instantiate or execute the machine readable instructions to configure a first medical imaging device, capture a first medical image of a patient using the first medical imaging device, analyze the first medical image, determine, based on an analysis of the first medical image, to capture a second medical image, configure at least one of the first medical imaging device or a second medical imaging device for the second medical image different from the first medical image, capture the second medical image, analyze the second medical image, and transmit the first medical image and the second medical image to an external device for processing and generating a next action with respect to the patient.
Example 2 includes example 1 including, wherein the first medical imaging device is located at a first location and the network interface is to transmit an instruction to capture the second medical image with the second medical imaging device at a second location.
Example 3 includes any of example 1 and example 2, wherein the network interface is to transmit an instruction to the second medical imaging device to determine whether, after capture of the second medical image by the second medical imaging device, a third medical image is to be captured by the second medical imaging device.
Example 4 includes any of examples 1-3, wherein the second medical image is captured by at least one of a magnetic resonance imaging system, a mammography system, or an ultrasound system.
Example 5 includes any of examples 1-4, wherein the second medical image is captured by at least one of a magnified imaging protocol, a spot examination protocol, or a contrast-enhanced mammography protocol.
Example 6 includes any of examples 1-5, wherein the apparatus is to, during analysis of the first medical image, determine if a positioning of a patient during the capture of the first medical image is correct.
Example 7 includes any of examples 1-6, wherein the apparatus is to, after determining that the positioning of the patient during the capture of the first medical image was not correct, present a notification on a display, the notification indicating for a technician to adjust the patient.
Example 8 includes any of examples 1-7, wherein the apparatus is to, during a subsequent operation to perform based on at least one of findings in the first medical image, patient breast density, malignancy score, BI-RADS score, and calculated risk score.
Example 9 includes any of examples 1-8, wherein the apparatus is to perform a determination of whether a late contrast-enhanced mammography (CEM) image is to be captured.
Example 10 includes any of examples 1-9, wherein when the apparatus determines that a late CEM image is to be captured, the apparatus is to capture a first CEM image and a second CEM image, analyze the first CEM image and the second CEM image with a dual energy technique, and determine if an amount of absorbed contrast is different between the first CEM image and the second CEM image.
Example 11 includes any of examples 1-10, wherein the network interface, machine readable instructions, and programmable circuitry are implemented in at least breast imaging protocol circuitry and breast imaging analysis circuitry, the breast imaging protocol circuitry to configure the first medical imaging device and the breast imaging analysis circuitry to analyze at least one of the first medical image or the second medical image.
Example 12 includes any of examples 1-11, wherein the apparatus is located on at least one of the first medical imaging device or the second medical imaging device.
Example 13 includes any of examples 1-12, wherein the apparatus is in communication with but located remotely from at least one of the first medical imaging device or the second medical imaging device.
Example 14 includes any of examples 1-13, wherein the apparatus generates a local imaging capture protocol based on availability of specific medical imaging devices at a specific medical location, the local imaging capture protocol different from a global imaging capture protocol.
Example 15 includes any of examples 1-14, wherein, based on the analysis of the first medical image, the apparatus determines not to capture the at least one second medical image, and transmits the first medical image to the external device.
Example 16 includes a non-transitory machine readable storage medium comprising instructions to cause programmable circuitry to at least configure a first medical imaging device, capture a first medical image of a patient using the first medical imaging device, analyze the first medical image, determine, in response to an analysis of the first medical image, to capture a second medical image, configure at least one of the first medical imaging device or a second medical imaging device different from the first medical image, capture the second medical image, analyze the second medical image, and transmit the first medical image and the second medical image to an external device for processing and generating a next action with respect to the patient.
Example 17 includes example 16 including, wherein the instructions are further to cause the programmable circuitry to, upon patient selection, configure at least one of the first medical imaging device and the second medical imaging device perform a recommended next medical image.
Example 18 includes a method comprising configuring, by implementing an instruction with a processor, a first medical imaging device, capturing, a first medical image of a patient using the first medical imaging device, analyzing the first medical image, determining, in response to an analysis of the first medical image, to capture a second medical image, configuring at least one of the first medical imaging device or a second medical imaging device for the second medical image different from the first medical image, capturing the second medical image, and transmitting the first medical image and the second medical image to an external device for processing and generating a next action with respect to the patient.
Example 19 includes example 18 including, wherein the first medical imaging device is located at a first location, further including transmitting an instruction to capture the second medical image with the second medical imaging device at a second location.
Example 20 includes any of example 18 and example 19, further including presenting a notification on a display, and configuring the first medical imaging device to repeat the first medical image.

The following claims are hereby incorporated into this Detailed Description by this reference. Although certain example systems, apparatus, articles of manufacture, and methods have been disclosed herein, the scope of coverage of this patent is not limited thereto. On the contrary, this patent covers all systems, apparatus, articles of manufacture, and methods fairly falling within the scope of the claims of this patent.

What is claimed is:

1. An apparatus comprising:
network interface;
machine readable instructions; and
programmable circuitry to at least one of instantiate or execute the machine readable instructions to:
configure a first medical imaging device;
capture a first medical image of a patient using the first medical imaging device;
analyze the first medical image;
determine, based on an analysis of the first medical image, to capture a second medical image;
configure at least one of the first medical imaging device or a second medical imaging device for the second medical image different from the first medical image;
capture the second medical image;
analyze the second medical image; and
transmit the first medical image and the second medical image to an external device for processing and generating a next action with respect to the patient.

2. The apparatus of claim 1, wherein the first medical imaging device is located at a first location and the network interface is to transmit an instruction to capture the second medical image with the second medical imaging device at a second location.

3. The apparatus of claim 2, wherein the network interface is to transmit an instruction to the second medical imaging device to determine whether, after capture of the second medical image by the second medical imaging device, a third medical image is to be captured by the second medical imaging device.

4. The apparatus of claim 1, wherein the second medical image is captured by at least one of a magnetic resonance imaging system, a mammography system, or an ultrasound system.

5. The apparatus of claim 1, wherein the second medical image is captured by at least one of a magnified imaging protocol, a spot examination protocol, or a contrast-enhanced mammography protocol.

6. The apparatus of claim 1, wherein the apparatus is to, during analysis of the first medical image, determine if a positioning of a patient during the capture of the first medical image is correct.

7. The apparatus of claim 6, wherein the apparatus is to, after determining that the positioning of the patient during the capture of the first medical image was not correct, present a notification on a display, the notification indicating for a technician to adjust the patient.

8. The apparatus of claim 1, wherein the apparatus is to, during a subsequent operation to perform based on at least one of findings in the first medical image, patient breast density, malignancy score, BI-RADS score, and calculated risk score.

9. The apparatus of claim 1, wherein the apparatus is to perform a determination of whether a late contrast-enhanced mammography (CEM) image is to be captured.

10. The apparatus of claim 9, wherein when the apparatus determines that a late CEM image is to be captured, the apparatus is to:
capture a first CEM image and a second CEM image;
analyze the first CEM image and the second CEM image with a dual energy technique; and
determine if an amount of absorbed contrast is different between the first CEM image and the second CEM image.

11. The apparatus of claim 1, wherein the network interface, machine readable instructions, and programmable circuitry are implemented in at least breast imaging protocol circuitry and breast imaging analysis circuitry, the breast imaging protocol circuitry to configure the first medical imaging device and the breast imaging analysis circuitry to analyze at least one of the first medical image or the second medical image.

12. The apparatus of claim 1, wherein the apparatus is located on at least one of the first medical imaging device or the second medical imaging device.

13. The apparatus of claim 1, wherein the apparatus is in communication with but located remotely from at least one of the first medical imaging device or the second medical imaging device.

14. The apparatus of claim 1, wherein the apparatus generates a local imaging capture protocol based on availability of specific medical imaging devices at a specific medical location, the local imaging capture protocol different from a global imaging capture protocol.

15. The apparatus of claim 1, wherein, based on the analysis of the first medical image, the apparatus determines not to capture the at least one second medical image, and transmits the first medical image to the external device.

16. A non-transitory machine readable storage medium comprising
instructions to cause programmable circuitry to at least:
configure a first medical imaging device;
capture a first medical image of a patient using the first medical imaging device;
analyze the first medical image;
determine, in response to an analysis of the first medical image, to capture a second medical image;
configure at least one of the first medical imaging device or a second medical imaging device different from the first medical image;
capture the second medical image;
analyze the second medical image; and
transmit the first medical image and the second medical image to an external device for processing and generating a next action with respect to the patient.

17. The non-transitory machine readable storage medium of claim 16, wherein the instructions are further to cause the programmable circuitry to, upon patient selection, configure at least one of the first medical imaging device and the second medical imaging device perform a recommended next medical image.

18. A method comprising:
configuring, by implementing an instruction with a processor, a first medical imaging device;
capturing a first medical image of a patient using the first medical imaging device;
analyzing the first medical image;
determining, in response to an analysis of the first medical image, to capture a second medical image;
configuring at least one of the first medical imaging device or a second medical imaging device for the second medical image different from the first medical image;
capturing the second medical image; and
transmitting the first medical image and the second medical image to an external device for processing and generating a next action with respect to the patient.

19. The method of claim 18, wherein the first medical imaging device is located at a first location, further including transmitting an instruction to capture the second medical image with the second medical imaging device at a second location.

20. The method of claim 18, further including:
presenting a notification on a display; and
configuring the first medical imaging device to repeat the first medical image.
